(19) United States
(12) Patent Application Publication
(10) Pub. No.: US 2013/0210057 A1
(43) Pub. Date: Aug. 15, 2013

(54) USE OF LABEL-FREE BIOSENSORS TO UNDERSTAND AND IDENTIFY TREATMENT FOR CANCER

(71) Applicant: CORNING INCORPORATED, (US)

(72) Inventors: Huayun Deng, Painted Post, NY (US); Ye Fang, Painted Post, NY (US); Joydeep Lahiri, Painted Post, NY (US); Shizhang Ling, Timonium, MD (US)

(73) Assignee: CORNING INCORPORATED, CORNING, NY (US)

(21) Appl. No.: 13/767,142

(22) Filed: Feb. 14, 2013

Related U.S. Application Data
(60) Provisional application No. 61/598,656, filed on Feb. 14, 2012.

(51) Int. Cl. C12Q 1/02

(52) U.S. Cl. CPC C12Q 1/025
USPC 435/29

(57) ABSTRACT

The disclosure relates to methods of using dynamic mass redistribution data obtained from cancer cells cultured on waveguide grating biosensors in the presence of agonists and in the presence of chemotherapeutic agents, for predicting effective chemotherapies for the treatment of cancer.

### CROSS-REFERENCE TO RELATED APPLICATIONS

This application claims the benefit of priority under 35 U.S.C. §119 of U.S. Provisional Application Ser. No. 61/598,656 filed on Feb. 14, 2012 the content of which is relied upon and incorporated herein by reference in its entirety.

### FIELD

The present disclosure describes methods related to label-free biosensors for decoding pathway abnormality, particularly PI3K pathway hyperactivation in solid tumors. The present disclosure also describes methods related to label-free biosensors for selecting chemotherapies to treat solid tumors, particularly tumors having PI3K pathway hyperactivation.

### BACKGROUND

Human cancer is considered to be a pathway dysregulated disease. Cancer cells have acquired the ability to outgrow surrounding cells, to induce changes in the architecture of the tissue in which they arise and to survive hostile environments that are hypoxic, hypoglycemic or lack growth factors. The ability of tumor cells to outgrow their neighboring cells is often driven by constitutive activation of downstream proteins. Mutations in three types of genes, oncogenes, tumor suppressor genes and stability genes, empower cancer cells with these abilities. Mutations in oncogenes and tumor suppressor genes directly promote net cell growth by stimulating cell birth or inhibiting cell death. Mutations in stability genes indirectly promote tumorigenesis by facilitating the accumulation of mutations at higher than normal rates, thereby allowing more rapid acclimatization of cancer cells to new microenvironments. Genetic studies over several decades have discovered a wide range of tumor-associated genes and their mutations, many of which occur in signaling proteins involved in a small number of pathways. Genetic mutations are often enriched in positive regulatory loops (gain of function), and methylated genes in negative regulatory loops (loss of function), leading to the disruption of the normal cooperative behavior of cells and thus promoting tumor phenotypes. Mutations in oncogenes result in gene products that are either constitutively active or active under conditions in which the normal gene product is inactive. Activating mutations include translocations, amplifications and point mutations that affect residues important for the regulation or activity of the gene product. In most cases, a mutation in one allele, paternal or maternal, is sufficient for activation. Tumor suppressor genes, on the other hand, are inactivated by mutations or epigenetic mechanisms. Both the maternal and paternal alleles must be inactivated to eradicate the inhibitory potential of tumor suppressor gene products.

Considering that it is only the genetic differences that unequivocally distinguish the cancer cell from the normal cell, it is not surprising that recent therapeutic advances are based on agents that specifically target the products of the genes that are mutated in cancer cells. In theory, the product of any gene frequently mutated in cancer cells provides a reasonable target for drug development. In practice, however, proteins that are activated by mutations (oncogene products) are better targets than proteins that are inactivated by mutations (tumor suppressor gene products). This is because it is unlikely that small molecules or antibodies can restore activity to a tumor suppressor gene product that has been inactivated by a mutation. Although it is possible that drugs could be developed against downstream pathways that are activated when a tumor suppressor gene is mutated, this approach is less direct and specific than a direct attack on an activated oncogene product.

Mutation-targeted therapies interact with proteins that are different in the cancer cell than in any normal cell. By virtue of somatic mutation, the amino acid sequence or the concentration of the mutant gene product in cancer cells is different from that in normal cells. Moreover, abnormal proteins activate downstream pathways under circumstances in which the pathways would be inactive in normal cells. Thus, mutation-targeted therapies selectively inhibit cancer cell growth. Small molecule inhibitors that target multiple kinases have shown promising results in clinical trials to gain approval by the FDA. Examples include: (1) sunitinib maleate which is an inhibitor of PDGFR-α and PDGFR-β, VEGFR1, 2 and 3, KIT, colony stimulating factor (CSF)-1, Fms-like tyrosine kinase 3 (FTL3) and rearranged-during-transfection (RET) oncogene product and is approved for the treatment of GIST after disease progression on, or intolerance to, imatinib mesylate, as well as for the treatment of metastatic renal cell carcinoma after it was shown to increase response and progression-free survival in such patients; (2) sorafenib tosylate which is also a multi-kinase inhibitor of c-RAF, v-raf murine sarcoma viral oncogene homolog B1 (BRAF), KIT, FTL3, VEGFR2 and 3, and PDGFR, and also proved beneficial for the treatment of metastatic renal cancer.

Mutation-targeted therapies offer tremendous potential for reducing morbidity and mortality from cancer. However, what cancer types or specific patients are likely to be responsive to such drugs cannot be predicted a priori and can be evaluated only through standard and relatively inefficient clinical trials. However, it is possible that retrospective analyses may identify specific mutations in tyrosine kinases that correlate with response, as occurred with gefitinib and erlotinib. Accordingly, the biological specificity and toxicity of a drug cannot be reliably inferred from biochemical experiments with a panel of related enzymes. Thus, companion diagnostics are essential for realizing the potential of mutation-targeted therapies. Companion diagnostics which identify and detect genetic, protein, or gene expression markers to predict whether a drug works or causes adverse effect in patients, has emerged as an exciting new field over the last few years. With the cost of new drug approval getting increasingly higher, pharmaceutical companies are beginning to explore companion tests in order to develop safer and more effective drugs. The value of companion diagnostic tests has already been demonstrated by a number of marketed products, such as, for example, HercepTest for Herceptin, K-RAS mutation tests for Erbitux and Vectibix, B-RAF mutation tests for Zelboraf (vemurafenib) which is used for treating BRAF-mutation-positive metastatic melanoma, and ALK fusion gene tests for Xalkori (crizotinib) which is used for treating patients with non-small-cell lung cancer driven by an ALK (anaplastic lymphoma kinase) fusion gene. Development of companion diagnostic tests has received great attention in recent years. These tests can simplify the drug discovery process, make clinical trials more efficient and informative, and can be used to individualize the therapy of cancer patients.

However, a variety of issues can compromise the effective use of companion diagnostic tests. Almost all companion diagnostic tests are based on genetic testing today. There are at least three requirements for an effective companion diagnostic test: availability of material suitable for testing; a set of mutations that are predictive of a therapeutic response; and an accurate and cost-effective technique for identifying mutations. The most commonly used methods in companion diagnostics today include immunohistochemistry, cytogenetics, FISH (fluorescent in situ hybridization), quantitative PCR (qPCR), DNA sequencing, indirect mutation analysis, mutation specific assays and microarrays.

Immunohistochemistry can detect small numbers of mutant-containing cells within a large background of normal cells, can be routinely performed in hospital labs, and is fast and cost effective. However, it is not quantitative. That is, the fraction of cells that express the antibody-reacted protein can be determined, but the level of protein within individual cells can only be crudely estimated, and it could be difficult to distinguish between expression levels due to gene amplification versus other, nongenetic causes.

Cytogenetics is quantitative and provides unambiguous evidence of genetic alterations. However, only a small number of cells can be analyzed as it is difficult to obtain large numbers of mitotic cells. The process is tedious and time consuming. It also requires significant experience to interpret data.

FISH is quantitative and provides unambiguous evidence of genetic alterations. Because interphase rather than mitotic cells are used, relatively large number of cells can be analyzed. However, FISH is time consuming and expensive, requires special equipment and significant experience to interpret data.

qPCR is very sensitive with large dynamic range; however, it requires careful controls to interpret data. Furthermore, results are highly dependent on sample quality. It also cannot distinguish between a lot of cells with a little transcript or a few cells with a lot of transcript.

DNA sequencing provides unequivocal evidence of somatic mutations. However, it does not detect some important mutations (e.g., deletions of an entire exon or amplifications). It is also insensitive, as detection requires the mutation to be present in at least 20% of all DNA molecules in the sample.

Indirect mutation analysis is cost effective and is in most cases more sensitive than sequencing when neoplastic cells represent a minor fraction of the total cells in a clinical sample. Some mutations can be difficult to be detected, even when presented in a major fraction of the cells. Extensive optimization may be required to detect mutations in individual genes or PCR fragments. In general, the presumptive mutation must be detected and precisely defined by a direct method such as sequencing.

Mutation specific assays are cost effective and are in most cases more sensitive than sequencing when neoplastic cells represent a minor fraction of the total cells in the clinical sample. However, they can only identify a small number of predefined mutations rather than any mutation that happens to be present in the cancer, and again often requires extensive optimization.

Microarrays allow massive, parallel, high-throughput interrogation of many genes. Patterns of gene expression can be useful for prognostication. Companion diagnostic tests generally need to interrogate only one or a few genes per sample and microarrays are inappropriate for this purpose. They require careful controls to ensure reproducibility.

Selection of the appropriate assay for the detection of genetic alterations depends on the type of mutation that must be detected. For example, when the target gene can be mutated in multiple different ways, direct or indirect sequencing methods should be used. On the other hand, if the identification of one or a small array of predefined mutations is the goal, then a mutation-specific method can be employed. If an increased copy number of the target gene, rather than a subtle change in amino acid sequence, is sought, then FISH or quantitative qPCR should be used. The type of material to be assessed can also influence the choice of assays. Samples containing a high fraction of neoplastic cells, such as those from the primary tumor or from microdissected lesions, can be used for direct sequencing. However, if the clinical sample contains a large fraction of normeoplastic cells, mutation-specific assays or indirect mutation analysis methods are advantaged over sequencing because they are more sensitive in these circumstances. Cost effectiveness is also an issue, though companion diagnostic testing will generally constitute only a small fraction of the total cost of treatment with a mutation-targeted agent.

### SUMMARY

The present disclosure describes companion diagnostic testing methods related to label-free biosensors for decoding pathway abnormalities in cancer, and for predicting effective chemotherapies to treat cancer. The present methods can be extended to any cancer, including solid tumors having diverse genetic or pathway abnormality. In embodiments, the disclosure provides methods using a label-free biosensor for cancer diagnostics and chemotherapy selection comprising:
a. providing an array of resonant waveguide grating biosensors;
b. culturing a cancer cell on at least one biosensor in the biosensor array;
c. treating the cultured cancer cell with at least one chemotherapeutic agent in the absence and presence of at least one agonist;
d. Collecting dynamic mass redistribution (DMR) signals from the cultured cancer cell treated with the at least one chemotherapeutic agent in the absence and presence of the at least one agonist in the biosensor array;
e. performing comparative analysis to generate a cancer cell fingerprint of the cultured cancer cell responding to the at least one chemotherapeutic agent in the absence and presence of the at least one agonist;
f. culturing a reference cell on at least one biosensor in the biosensor array, treating the cultured reference cell with the at least one chemotherapeutic agent in the absence and presence of the at least one agonist, collecting the DMR signals from the cultured reference cell treated with the at least one chemotherapeutic agent in the absence and presence of the at least one agonist in the biosensor array, so the cultured reference cell generates a reference cell fingerprint responding to the at least one chemotherapeutic agent in the absence and presence of the at least one agonist;
g. comparing the reference cell fingerprint to the cancer cell fingerprint;
h. treating the cancer cell and the reference cell with at least one reference compound, wherein the reference compound generates a reference compound control fingerprint;
i. comparing the cancer cell fingerprint with the reference cell fingerprint, and the reference compound control fingerprint; and,
j. using the compared data from the cancer cell fingerprint, the reference cell fingerprint and the reference compound control fingerprint to determine a chemotherapy regimen to treat the cancer represented by the cancer cells.

### BRIEF DESCRIPTION OF THE DRAWINGS

FIG. 1 is a graph illustrating Ca²⁺ mobilization induced by G protein-coupled receptor (GPCR) agonists in paired isogenic cell lines, PCT116 parental (squares) and HCT116-PTEN Null (diamonds) cell line. The fold increase in fluorescence due to Ca²⁺ mobilization, as measured using FLIPR (Fluorescent Imaging Plate Reader), was plotted as a function of GPCR agonist.

FIG. 2 is a graph illustrating cAMP accumulation induced by GPCR agonists. The percentage of cAMP level after normalized to the forskolin-induced cAMP increase was plotted as a function of GPCR agonists. PCT116 parental (squares) was compared with HCT116-PTEN Null (diamonds) cell line.

FIG. 3 represents a heat map of Dynamic Mass Redistribution signals induced by GPCR agonists in PCT116 parental (Parental) and HCT116-PTEN Null (PTEN Null) cell lines. The real responses, as recorded as shift in resonant wavelength (picometer, pm) of the biosensor, at six time points (3 min, 5 min, 9 min, 15 min, 30 min and 45 min) poststimulation, for each compound-induced DMR were extracted and used as the basis for similarity analysis. The contrast was set to be −200 to 200 pm. The saturated (black) areas mean a response whose absolute value is equal to and greater than 200 pm. The heat map was generated using unsupervised Ward hierarchical clustering. Only DMR signal that contains at least one data point greater than 80 pm was included in the analysis.

FIG. 4 is a bar graph illustrating quantitative real time PCR confirmed that the expression pattern of a panel of GPCRs is largely identical in the two isogenic cell lines. Almost all GPCRs, had mRNA levels which were found to be within 2 fold after PTEN deletion. For each cell line, the cycle threshold value of each gene was normalized to the control gene, Hypoxanthine phosphoribosyltransferase 1.

FIG. 5a, b and c are graphs illustrating DMR characteristics of 10 μM adenosine in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 6 a, b and c are graphs illustrating DMR characteristics of 10 μM epinephrine in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 7 a, b and c are graphs illustrating DMR characteristics of 10 μM forskolin in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 8 a, b and c are graphs illustrating DMR characteristics of 10 μM SFLLR in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 9 a, b and c are graphs illustrating DMR characteristics of 10 μM SLIGKV in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 10 a, b and c are graphs illustrating DMR characteristics of 10 μM ATP in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 11 a, b and c are graphs illustrating DMR characteristics of 10 μM neurotensin in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 12 a, b and c are graphs illustrating DMR characteristics of 1 μM epidermal growth factor (EGF) in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 13 a, b and c are graphs illustrating DMR characteristics of 1 μM insulin growth factor-1 (IGF1) in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 14 is a photograph of a Western blot. FIG. 14 illustrates that multiple pathways are transactivated by multiple GPCR agonists. Cells were serum-starved overnight and then treated with each of 5 GPCR agonists for 5 minutes in serum-free medium. Proteins in the cell lysates were resolved on 4-12% SDS-PAGE gel, followed by Western blotting with each of these indicated antibodies. SFM represents serum starvation overnight (0% serum) using the serum-free medium. Compound treatment: 5 min. The concentration for each compound was SFLLR=1 μM, SLIGKV=1 μM, Neurotensin=μuM, S1P=2.5 μM, and LPA=2.5 μM. P=phospho and T=total in the labels.

FIG. 15 a and b are graphs illustrating differences in potency and efficacy of SFLLR to trigger DMR signals in between the two isogenic cell lines (HCT116 parental cell line; HCT116-PTEN Null cell line). Both the early DMR amplitude (a) and the late DMR amplitude (b) were plotted as a function of SFLLR doses.

FIG. 16 a, b, c, d, e and f are graphs illustrating DMR characteristics of a panel of kinase inhibitor drugs in both HCT116 parental (Control) cell line and HCT116-PTEN null (PTEN null) cell lines. (a) Everolimus, (b) Sirolimus, (c) Temsirolimus, (d) Erlotinib, (e) Geftinib, and (f) Imatinib. Each was assayed at 16 μM. The data represents mean±s.d. from 2 independent measurements, each in duplicate (n=4).

FIG. 17 a, b, c, d and e are graphs illustrating DMR characteristics of a panel of kinase inhibitor drugs in both HCT116 parental (Control) cell line and HCT116-PTEN null (PTEN null) cell lines. (a) lapitinib, (b) pazopanib, (c) nilotinib; (d) sorafenib, (e) sunitinib. Each was assayed at 16 μM. The data represents mean±s.d. from 2 independent measurements, each in duplicate (n=4).

FIG. 18 a, b and c are graphs illustrating the dose-dependent alteration of the late DMR of IGF1 in the two isogenic cell lines (HCT116 parental cell line; HCT116-PTEN Null cell line) by different kinase inhibitors: (a) erlotinib; (b) geftinib; (c) imatinib. The DMR amplitudes 50 min postsimulation with 2 nM IGF1 were plotted as a function of kinase inhibitors. The kinase inhibitors at different doses were used to pretreat the cells for about 1 hr, before IGFR1 was added.

FIG. 19 is a heat map of fifty-five NCI60 cell lines. The heat map was generated based on the DMR signals of a small panel of GPCR agonists. The real responses, as recorded as shift in resonant wavelength (pm), at six time points (3 min, 5 min, 9 min, 15 min, 30 min and 50 min) poststimulation, for each compound-induced DMR was extracted and used as the basis for similarity analysis. The contrast was set to be −200 to 200 pm. The saturated areas (black) mean a response whose absolute value is equal to and greater than 200 pm. The heat map was generated using unsupervised Ward hierarchical clustering.

FIG. 20 is a heat map of fifty-five NCI60 cell lines. The heat map was generated based on the DMR signals of a small panel of reference compounds that intervene intracellular targets. The real responses, as recorded as shift in resonant wavelength (pm), at six time points (3 min, 5 min, 9 min, 15 min, 30 min and 50 min) poststimulation, for each compound-induced DMR was extracted and used as the basis for similarity analysis. The contrast was set to be −200 to 200 pm. The saturated color means a response whose absolute value is equal to and greater than 200 pm. The heat map was generated using unsupervised Ward hierarchical clustering.

### DETAILED DESCRIPTION

The present disclosure describes companion diagnostic tests which utilize label-free biosensors for decoding pathway abnormalities. The present disclosure also describes methods related to the use of label-free biosensors for selecting chemotherapies to treat cancer. The methods can be extended to the use of label-free biosensors to decode or diagnose other specific pathway or genetic abnormalities, and to determine appropriate chemotherapy regimens for the treatment of cancer.

In particular, the disclosure provides analysis related to PI3K pathway hyperactivation in cancers having PI3K pathway hyperactivation. These cancers can be solid tumor-generating cancers.

In embodiments the biosensor is a resonant waveguide grating biosensor contained in a well of a microtiter plate or a multiwell plate, or within a device which contains more than two separate wells each having at least one biosensor. When the biosensor is contained in a well of a microtiter plate having more than one well, the biosensor can be an “array” or a “biosensor array”. The biosensor array can contain microfluidic devices so that fluids can flow from one well to another well. For example, chemotherapy agents or test compounds (agonists, or reference compounds) can be introduced to a well containing a biosensor under a specific condition (e.g., perfusion). The biosensor can be a resonant waveguide grating biosensor. Surface plasmon resonance biosensors can also be used.

In embodiments, cells can be cultured on the biosensors in wells. Cultured cells can be primary cells or cell lines. Primary cells can be cultured from normal tissues or from abnormal tissues. Primary cells can be cancer cells or non-cancer cells. In embodiments, primary cell culture can be established by removing cells from a patient and allowing the cells to grow in a suitable media at a suitable temperature and in other conditions suitable for cell culture. In embodiments, primary cells can be cultures from cancerous tissue. In embodiments, primary cells from solid tumors can be cultured. For cell cultures from solid tumors specimen collection as soon after resection as possible is an important factor for obtaining cell suspensions with high viability as there is a decline in the viability of harvested cells as the longer the tissue remains ex vivo. Cells from solid tumors or healthy tissues can be isolated by first mincing surgical specimen into small pieces to remove red blood cells during the initial washing steps, followed by progressive enzymatic dissociation or physical dissociation (e.g., centrifugation, ultrasound treatment, or vertexing). Appropriate choice of enzyme, incubation time, temperature, and concentration for optimal digestion are required to obtain the best tissue dissociation without excessive destruction. Collagenase digestion is known to be less harmful to epithelial cells than is digestion with other enzymes (such as trypsin) and can give cell suspensions with high yields and viabilities. Sequential filtration of collagenase digest through a series of sieves with decreasing mesh sizes removes tubular fragments and glomeruli, and leaves material that yields outgrowth of cells, such as renal epithelial cells. Alternatively, Percoll density gradient centrifugation, or complex microdissection or immunomagnetic techniques can be used to establish primary culture of tumor cells. For solid tumor tissues having stromal fibroblast which typically grow quite rapidly in vitro, stromal deletion approaches such as Miltenyi EpCAM microbead-based fractionation method can be deployed to remove stromal fibroblast cells.

In embodiments, immortalized cell lines can also be used for detailed testing, however, when cancer cells are immortalized, they may undergo dedifferentiation (i.e., the loss of the original cell specificity) as a result of successful passages in vitro. As a result, cell lines may possess characteristics that are not present in the tissue of origin due to the immortalization process.

In embodiments, cultured cells can be treated with at least one chemotherapeutic agent. Treating of cells with a chemotheraputic agent may occur in the absence and presence of an agonist. Treatment can be performed in a single step or a sequential multi-step manner. The chemotherapy treatment can precede or follow an agonist treatment.

In embodiments where an agonist is present, the methods use a set of agonists to form an effective agonist panel to provide data related to a wide range of receptors and pathways. This can be made possible by the polypharmacology of the agonists used. The agonist panel can be selected from epinephrine for adrenergic receptors, histamine for histamine receptors, acetylcholine for muscarinic receptors, dopamine for dopamine receptors, LPA (Lysophospholipid) for LPA receptors, S1P (sphingosine-1-phosphate) for S1P receptors, adenosine for adenosine receptors, SFLLR for protease activating receptor-1 (PAR1), SLIGKV for PAR2, and ATP for purinergic P2Y receptors. Alternative agonists such as synthetic agonists for these receptors can also be used. Epinephrine can activate almost all 9 adrenergic receptors (alpha1A, C and D, alpha2A, B, C, and beta1, 2, 3-adrenergic receptors), while dopamine can activate all five dopamine receptors, histamine activates all four histamine receptors, LPA activates at least five LPA receptors, adenosine activates four adenosine receptors, S1P activates at least four S1P receptors, ATP activates at least three P2Y receptors.

In embodiments, the agonist panel comprises a plurality of agonists including agonists for distinct classes of GPCRs, and growth factor receptors. The choice of agonists is based on (1) polypharmacology of the agonist, wherein the greater the polypharmacology the better the agonist is; and (2) the ubiquitous expression of the receptor family in different tissues, wherein the wider expression of the receptor family in different tissues the better the agonist is. Tissue-specific receptor-agonist pair can also be included for specificity testing. For example, neurotensin receptor agonists can be included when tumors from guts are analyzed. Growth factor receptors can be selected from EGFR, IGF1R, where are more ubiquitously expressed in different types of cells than other growth factor receptors.

In embodiments, the plurality of agonists comprises at least: (1) an agonist for a Gq-coupled receptor; (2) an agonist for a Gs-coupled receptor; (3) an agonist for a Gi-coupled receptor; and (4) an agonist for a growth factor receptor. In embodiments the plurality of agonists also includes one or more of: epinephrine, histamine, acetylcholine, dopamine, ATP, S1P, or LPA. In embodiments, reference cell comprises a cancer cell line derived from the same origin of the solid tumor cell tested. For example, the reference cell lines relevant for comparing against colorectal solid tumor cells are HCT116 parental and PTEN null cell lines (both available for Horizon Discovery Ltd., Cambridge, UK). In embodiments, reference cell comprises a healthy cell that is obtained from the same tissue of the solid tumor cell tested.

In embodiments, DMR signals are collected in real time. Alternatively, responses at a predetermined time point can be collected. For example, responses at 3 min, 5 min, 9 min, 15 min, 30 min, or 45 min poststimulation (with an agonist or a polarity of agonists in an array) can be collected.

In embodiments, the DMR signals that have been collected, in the presence and absence of an array of agonists (or at least one agonist), and in the presence (and absence, for control) of a chemotherapeutic agent, taken together, form a “fingerprint” for that chemotherapeutic agent. For example, the DMR signals of a cancer cell responding to a chemotherapeutic agent, in the presence of an agonist which may be a Gq-coupled receptor, an agonist for Gs-coupled receptor, an agonist for a Gi-coupled receptor, an agonist for a growth factor receptor, epinephrine, histamine, acetylcholine and dopamine or any of the agonists shown in Table 1, provides an array of data about the cell's response, with respect to each of these agonists, and the effect of the chemotherapeutic agent on the cell's response with respect to each of the agonists. This array of DMR data is a fingerprint that can be used to describe the cell's response to the chemotherapeutic agent. This fingerprint is a cancer cell fingerprint. This cancer cell fingerprint can be used to predict the effectiveness of the particular chemotherapeutic agent to affect the cell in a desirable manner. In embodiments, the cancer cell fingerprint can be used to determine a chemotherapy regimen to treat the cancer represented by the cancer cells. The fingerprint can be presented in the form of a heat map generated based on similarity analysis of the chemotherapy-induced responses in the absence and presence of the agonist panel for cultured cancer cells (and cultured “normal” cells, for control).

In embodiments, the cells can be cultured cell lines. In additional embodiments the cells can be primary cells harvested from biopsied tissue or other tissue removed from a patient. In embodiments, the cells can be primary cells derived from a solid tumor.

In embodiments, a reference cell fingerprint can be generated based on data obtained from a reference cell. The reference cell line can be an immortalized cell line having similar origin to the cancer cells, or can be primary cells harvested from non-tumor cells. In embodiments, the reference cells may be primary cells or cell lines which provide the reference cell control fingerprint data to compare against the fingerprint generated with the cultured cancer cells. In embodiments, the reference cell fingerprint can be compared to the cancer cell fingerprint. In embodiments, the reference cell line is, for example, colon cancer cell line HCT116 vs HCT116-PTEN null cell lines. The HCT116-PTEN Null cell line contains a single PTEN gene deletion via state-of-the-art technology (e.g., isogenic gene deletion cell lines from Horizon Discovery Ltd.). Alternatively, the isogenic cell line can be the parental cell line bearing a mutation of a single gene, such as constitutively activated K-RAS.

In embodiments, the method may include gathering DMR data from cultured cells in the presence of a reference compound. In embodiments, reference compounds are compounds which have known cell effects, and which provide additional data to assist in understanding the data obtained from the cancer cell treated with the chemotherapeutic agent. In embodiments, the reference compound is known to trigger label-free dynamic mass redistribution signal in many, if not all, cells including cancerous cell lines, or primary cells or tissue cells. In embodiments, the reference compound may be forskolin for activating adenylyl cyclases, TBB (4,5,6,7-Tetrabromobenzotriazole) for inhibiting casein kinases, LY294002 for inhibiting PI3 kinases, Y27632 for Rho kinases, ODN2006 for activating Toll-like receptor-9 (TLR9). In embodiments, DMR data obtained from treating cultured cells (either cancer cells or reference cells) can generate a reference compound control fingerprint. This reference compound control fingerprint can be compared against the fingerprint generated from the cultured cancer cells treated with chemotherapeutic agents, and compared to the reference cell fingerprint treated with chemotherapeutic agents, to augment a diagnostician's understanding of the cells, and to assist in determining an appropriate chemotherapeutic agent, or combination of agents, to use to treat cancer.

In an aspect (1), the disclosure provides a method of using a label-free biosensor for cancer diagnostics and chemotherapy selection comprising: (a) providing an array of resonant waveguide grating biosensors; culturing one or more cancer cells on at least one biosensor in the biosensor array; (b) treating the cultured cancer cell with at least one chemotherapeutic agent in the absence and presence of at least one agonist; (c) collecting dynamic mass redistribution (DMR) signals from the cultured cancer cell treated with the at least one chemotherapeutic agent in the absence and presence of the at least one agonist in the biosensor array; and, (d) performing comparative analysis to generate a cancer cell fingerprint of the cultured cancer cell responding to the at least one chemotherapeutic agent in the absence and presence of the at least one agonist.

In an aspect (2), the disclosure provides the method of aspect 1, wherein the at least one agonist is selected from the group consisting of an Gq-coupled receptor agonist, a Gs-coupled receptor agonist, a Gi-coupled receptor agonist, a growth factor receptor agonist, epinephrine, histamine, acetylcholine and dopamine

In an aspect (3), the disclosure provides the method of aspect 2 wherein the Gq-coupled receptor agonist is histamine.

In an aspect (4), the disclosure provides the method of aspect 2 wherein the Gq-coupled receptor agonist is acetylcholine.

In an aspect (5), the disclosure provides the method of aspect 2 wherein the Gs-coupled receptor agonist is epinephrine.

In an aspect (6), the disclosure provides the method of aspect 2 wherein the Gi-coupled receptor agonist is dopamine.

In an aspect (7), the disclosure provides the method of aspect 2 wherein the specific pathway is the PI3k/Akt pathway.

In an aspect (8), the disclosure provides the method of aspect 1 wherein the cancer cell is an immortalized cancer cell.

In an aspect (9), the disclosure provides the method of aspect 2 wherein the cancer cell is an immortalized cancer cell.

In an aspect (10), the disclosure provides the method of aspect 1 wherein the cancer cell is a primary cell.

In an aspect (11), the disclosure provides the method of aspect 2 wherein the cancer cell is a primary cell.

In an aspect (12), the disclosure provides the method of aspect 1 wherein the cancer cell is a primary cell derived from a solid tumor.

In an aspect (13), the disclosure provides the method of aspect 2 wherein the cancer cell is a primary cell derived from a solid tumor.

In an aspect (14), the disclosure provides the method of aspect 1 further comprising culturing a reference cell on at least one biosensor in the biosensor array, wherein the reference cell generates a reference cell fingerprint.

In an aspect (15), the disclosure provides the method of aspect 14 further comprising comparing the reference cell fingerprint to the cancer cell fingerprint.

In an aspect (16), the disclosure provides the method of aspect 1 further comprising treating the cultured cell with at least one reference compound, wherein the reference compound generates a reference compound control fingerprint.

In an aspect (17), the disclosure provides the method of aspect 15 wherein the reference compound is selected from the group consisting of forskolin, 4,5,6,7-tetrabromobenzotriazole, LY294002 and ODN2006.

In an aspect (18), the disclosure provides the method of aspect 15 further comprising comparing the cancer cell fingerprint with the reference compound control fingerprint.

In an aspect (19), the disclosure provides the method of aspect 1 further comprising using the cancer cell fingerprint to determine a chemotherapy regimen to treat the cancer represented by the cancer cells.

In an aspect (20), the disclosure provides a method of using a label-free biosensor for cancer diagnostics and chemotherapy selection comprising:
a. providing an array of resonant waveguide grating biosensors;
b. culturing one or more cancer cells on at least one biosensor in the biosensor array;
c. treating the cultured cancer cell with at least one chemotherapeutic agent in the absence and presence of at least one agonist;
d. Collecting dynamic mass redistribution (DMR) signals from the cultured cancer cell treated with at least one chemotherapeutic agent in the absence and presence of the at least one agonist in the biosensor array;
e. performing comparative analysis to generate a cancer cell fingerprint of the cultured cancer cell responding to the at least one chemotherapeutic agent in the absence and presence of the at least one agonist;
f. culturing one or more reference cells on at least one biosensor in the biosensor array, treating the cultured reference cell with the at least one chemotherapeutic agent in the absence and presence of the at least one agonist, collecting the DMR signals from the cultured reference cell treated with the at least one chemotherapeutic agent in the absence and presence of the at least one agonist in the biosensor array, so the cultured reference cell generates a reference cell fingerprint responding to the at least one chemotherapeutic agent in the absence and presence of the at least one agonist;
g. comparing the reference cell control fingerprint to the cancer cell fingerprint;
h. treating the cultured cancer cell and the cultured reference cell with at least one reference compound, wherein the reference compound generates a reference compound control fingerprint;
i. comparing the cancer cell fingerprint with the reference cell fingerprint and the reference compound control fingerprint; and,
j. using the compared data from the cancer cell fingerprint, the reference cell fingerprint and the reference compound control fingerprint to determine a chemotherapy regimen to treat the cancer represented by the cancer cells.

### EXAMPLES

### 1. Materials and Methods

### 1.1 Cell Lines and Cell Culture Medium

Corning Epic® cell assay 384-well microplates that have a resonant waveguide grating optical biosensor in each well, are cell culture compatible, and fibronectin-coated were obtained from Corning Inc. (Cat#5042) (Corning Inc., NY).

A pair of isogenic human colorectal adenocarcinoma cell lines, HCT116 parental and PTEN null cell lines (reference cell lines), were licensed to Corning from Horizon Discovery Ltd. (Cambridge, UK). The cell culture medium was as follows: McCoy's 5a medium (modified, lx, Invitrogen) was supplemented with 10% FBS, 4.5 g/liter glucose, 1.5 mM glutamine, and 1% penicillin and streptomycin for HCT116 parental and PTEN null cell lines.

### 1.2 Calcium Flux Assay

FLUO-4 DIRECT™ Calcium Assay Kit was purchased from Invitrogen (Irvine, Calif.) (Starter pack, Cat. no. F10471). HCT116 parental or HCT116 PTEN null cells (25000 cells/well) were seeded in poly-D-lysine coated 384-well black wall, clear bottom plates (Cat#3845) (Corning Inc., NY), cultured overnight at 37° C. The next day, Ca²⁺ flux assay was performed following manufacturer's instruction and fluorescence ratio (340 nm/380 nm) was measured on Functional Drug Screening System (FDSS) (Hamamatsu Photonics, Japan). This measurement is also known as FLIPR (Fluorometric Imaging Plate Reader). Briefly, compounds were prepared as 10× stock (final concentration 10 μM) in the Ca²⁺ assay buffer in a 384-well compound plate (Cat#3565) (Corning Inc., NY). Cell culture medium was not removed; cells were not washed. Baseline fluorescence was measured for 15 seconds before compound addition by onboard liquid handler robot. After compound addition, fluorescence was measured for 2 minutes.

1.3 cAMP Assay

HCT116 parental cells or HCT116 PTEN null cells (reference cell lines) were seeded at a density of 15000 cells/well in 384-well white wall, clear bottom, and cell culture treated plates (BD Bioscience, Franklin Lakes, N.J., Cat#354660). Cells were cultured in the McCoy's 5a complete growth medium overnight. The next day cAMP-Glo assay was performed according to manufacturer's instruction (Promega, Cat# V1502). Briefly, cell culture medium was removed by flipping the plate. 7.5 μL of each compound (10 μM in induction buffer) (Cat#3565) (Corning Inc., NY) was added to each well by liquid handler robot (CyBio) for 15 minutes incubation before additions of lysis buffer, PKA kinase assay buffer, and substrate. Luminescence was measured using Tecan Salim II plate reader.

### 1.4 Western Blotting

Antibodies against EGFR, phospho-EGFR (Tyr1068), Akt, phospho-Akt (Ser473), p44/42 MAPK (Erk1/2), phospho-p44/42 MAPK (Erk1/2) (Thr202/Tyr204), phospho-Stat3 (Ser727), and PTEN were obtained from Cell Signaling Technology, Inc. (Beverly, Mass.). Rabbit monoclonal antibody against phospho-c-Src PY529 was obtained from Epitomics (Burlingame, Calif.). Monoclonal anti-β-actin antibody was obtained from Sigma (St. Louis, Mo.).

HCT116 parental and PTEN null cells were cultured in 6-well plate (Cat#3335) (Corning, N.Y.) to reach around 80% confluency, and then serum starved overnight before pretreatment with each of various inhibitors for 2 hours. After pretreatment, cells were further treated with Gq-coupled receptor agonist, Gs-coupled receptor agonist, Gi-coupled receptor agonist or a growth factor receptor agonist for 5 minutes. Cells were lysed in RIPA lysis buffer (Cat# R 0278) (Sigma-Aldrich, St. Louis, Mo.) supplemented with protease inhibitors tablet (Roche Diagnostic Systems, Branchburg, N.J.) and phosphatase inhibitor cocktail I and II (Sigma-Aldrich, St Louis, Mo.) and harvested by scraping using rubber policeman on ice. The detergent-resistant pellets were removed after centrifugation (15000 g, 15 minutes) at 4° C. to generate whole cell lysate. Protein concentration for each cell lysate was determined by the Lowry protein assay (Bio-Rad Laboratories, Hercules, Calif.). Equal amount of total protein for each sample was mixed with Laemmli sample buffer (62.5 mM Tris-HCl, pH 6.8, 2% SDS, 10% glycerol, 0.1M DTT and 0.01% bromophenol blue) and then 40 μg of total protein for each sample were resolved on 4-12% Criterion XT Bis-Tris polyacrylamide precast gel Cat#345-0125) (Bio-Rad Laboratories, Hercules, Calif.), and transferred to nitrocellulose membrane.

The membrane was blocked with PBS supplemented with 0.1% Tween 20 and 5% nonfat milk for 1 h at room temperature, and then probed with each of the indicated primary antibody for 1 h at room temperature (EGFR, p42/44 ERK1/2, PI3K, PTEN, or β-actin) or overnight (phospho-EGFR, phospho-p42/44 ERK1/2, phospho-PI3K, or phospho-c-Src PY529) at 4° C. followed by HRP-conjugated appropriate secondary antibodies for 45 minutes at room temperature (Santa Cruz Biotech, Santa Cruz, Calif.). Signals from immunoreactive bands were developed by enhanced chemiluminescence (ECL) plus kit (GE Health Care, Piscataway, N.J.) according to the manufacturer's instructions and detected on KODAK X-OMAT 2000A Processor.

### 1.5 Optical Biosensor System and Cell Assays

EPIC® beta version wavelength interrogation system (Corning Inc., NY) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. Also EPIC® commercial systems were used, with a liquid handler accessory attached to EPIC® reader system. Also, an Epic® RWG imager can be used.

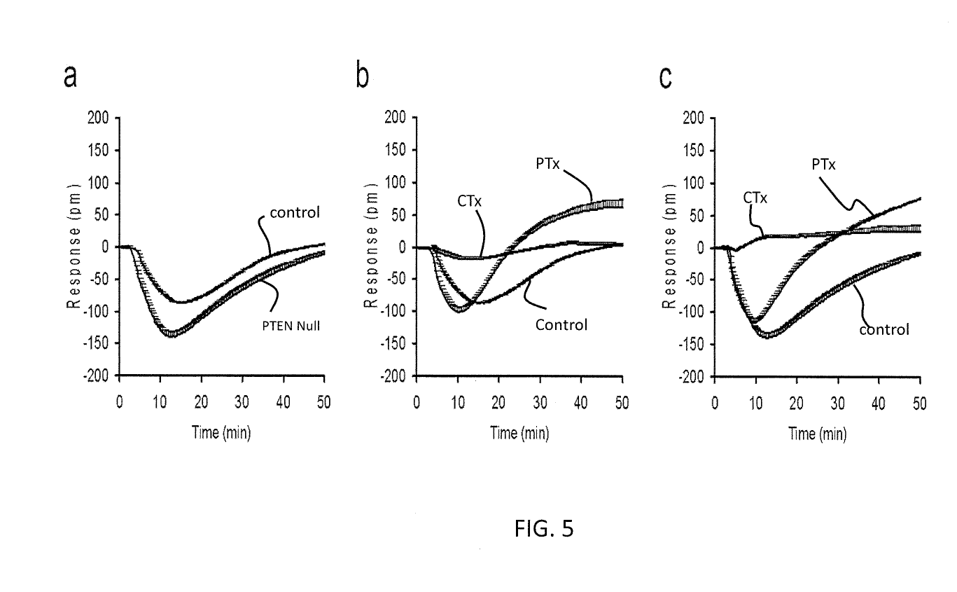

The resonant waveguide grating (RWG) biosensor is capable of detecting minute changes in local index of refraction near the sensor surface and the waveguide thin film having gratings provide a cell culture surface. Since the local index of refraction within a cell is a function of density and its distribution of biomass (e.g., proteins, molecular complexes), the biosensor exploits its evanescent wave to non-invasively detect ligand-induced Dynamic Mass Redistribution (DMR) in native cells. When a cell is stimulated, it responds. That response is a redistribution of biomass in the cell. Upon stimulation, proteins, protein complexes, and/or organelles in the cells move. The evanescent wave extends into the cells and exponentially decays over distance, leading to a characteristic sensing depth of ˜150 nm. Any optical response mediated through receptor activation only represents an average over the portion of the cell that the evanescent wave is sampling. The aggregation of many cellular events downstream to the receptor activation leads to a DMR signal having characteristic kinetics and amplitudes. These are ligand-induced DMR signals, as shown, for example, in FIGS. 5-13.

For biosensor cellular assays, cells were typically grown using ˜1 to 2×10⁴ cells per well at passage 3 to 15 suspended in 50 μl of culture medium in 384-well Corning EPIC cell assay microplates (Cat #5042) (Corning Inc., NY) and were cultured at 37° C. in an incubator with air/5% CO₂ for ˜1 day. Confluency for all cells at the time of assays was ˜95% to 100%. Compound solutions were made by diluting the stock compound solutions with the 1×HBSS-HEPES buffer (Hank's balanced salt solution, plus 20 mM HEPES, pH 7.1). Compound solutions were transferred into a 384-well polypropylene compound storage plate (Cat#3656) (Corning Inc., NY) to make a source (or compound) plate. In parallel, the cells were washed thrice with the HBSS-HEPES buffer and maintained in 30 μl of the HBSS-HEPES to make an assay (or cell) plate. Both the source and assay plates were then loaded and incubated in the hotel of the EPIC® reader system to establish equilibrium. After ˜1 hr of incubation, the baseline wavelengths of all biosensors in the assay plate (containing cells) were recorded and normalized to zero. Afterwards, a 2 to 10 minute continuous recording was carried out to establish a baseline, and to ensure that the cells were at a steady state. Cellular responses in each well of the 384-well assay microplate were then triggered by adding 10 μl of each compound solution from the source plate into the assay plate using the on-board liquid handler.

To study G protein coupling for compounds that are ligands of GPCRs, cells were seeded in 384-well fibronectin-coated Corning EPIC® cell assay microplates (Cat#5042) (Corning Inc., NY) 4-6 hours after seeding, cells were treated with cholera toxin (CTX, 100 ng/ml) or pertussis toxin (PTX, 100 ng/ml) overnight (12-16 hours) before cells were stimulated with a library of compounds.

CTX catalyzes the ADP ribosylation of the G_{sα} subunit of the heterotrimeric G protein, resulting in constitutive cAMP production. PTX catalyzes the ADP-ribosylation of G_{iα} subunit, thereby leading to the prevention of the interaction of the G_{iα} with GPCRs and inhibiting adenylyl cyclase.

To study the influence of compounds on a response relevant to chemotherapy, a second stimulation with an agonist at a fixed dose (typically at EC80 or EC100) was applied. The resonant wavelengths of all biosensors in the microplate were normalized again to establish a second baseline, right before the second stimulation. The two stimulations were usually separated by ˜1 hr.

All assays were carried out at a controlled temperature (28° C.). At least two independent sets of experiments, each with at least three replicates, were performed. The assay coefficient of variation was found to be <10%.

### Example 1

### PI3K Pathway Hyperactivation and Cancers

PI3K catalyzes the production of the second messenger phosphatidylinositol 3,4,5-trisphosphate (PIP3), thereby recruiting and activating several downstream kinases. PI3K and its most prominent effector, Akt (also known as PKB), regulate cell viability, metabolism, motility, and proliferation and are extensively implicated in tumorigenesis. Constitutive activation of the PI3K/Akt signaling pathway, often referred to PI3K pathway hyperactivation, is one of the most commonly occurring mutations in all cancers. The PI3K pathway hyperactivation can be achieved through many different cellular mechanisms, for example, but not limited to, (1) overexpression of certain targets including EGFR and casein kinase 2; (2) expression of gain-of-function mutations of certain targets, including certain K-RAS, B-Raf, c-Raf, and PI3K; (3) deletion or inactivation of negative regulators of the PI3K/AKT pathway.

The main negative regulator of the PI3K/Akt pathway, the lipid phosphatase and tensin homolog (PTEN), is frequently inactivated in human cancer as result of various genetic lesions, which ultimately result in decreased or absent PTEN protein expression and activity. PTEN deficiency in mice replicates the tumor spectrum observed in humans, including T cell malignancies, T cell-specific deletion of PTEN results in lymphoma-induced death. PTEN inactivation and consequent PI3K/Akt pathway aberrant activation may arise from mechanisms other than those targeting PTEN gene integrity. PTEN C-terminal phosphorylation appears to stabilize the PTEN protein by preventing its ubiquitination and proteasome degradation while decreasing PTEN phosphatase activity. The serine/threonine protein kinase casein kinase 2 (CK2) has been linked to PTEN phosphorylation. CK2 overexpression is observed in human solid tumors and is essential for multiple myeloma cell survival. Moreover, transgenic mice with targeted expression of CK2 in T cells develop lymphomas.

In embodiments, paired isogenic cancer cell lines, HCT116 parental cell line and its HCT116-PTEN-Null cell line, were used to demonstrate the ability of label-free whole cell assays to decode the hyperactivation of PI3K pathways as a result of PTEN deletion, as well as its effect on GPCR signaling. The ability to differentiate these paired cell lines (the HCT116 parental cell line and its PTEN null) in their responses to known kinase inhibitor drugs, demonstrates the feasibility of label-free whole cell assays for cancer diagnostics and chemotherapy selection. These techniques can also be used for discovering drugs that specifically modulate responses of mutation-bearing cell lines to treatment with relevant agonists.

### Example 2

### Ca²⁺ Mobilization Screening Using the Paired Isogenic Cell Lines

To screen endogenous receptors and their coupled signaling pathways, we first assembled a GPCR agonist focused library (Table 1). The library consists of 120 GPCR agonists covering over 150 known GPCRs, based on their known pharmacology including polypharmacology. For small molecule chemical agonists, the dose used was 10 micromolar. For peptide agonists, the dose used was 1 micromolar. For lipid agonists, the dose used was also 1 micromolar. For both peptides and lipids, these agonists were suspended in a solution containing 0.05% bovine serum to increase their stability.

TABLE 1
GPCR agonists and their paired target receptors.

| Compound | Targets |
|---|---|
| Serotonin | 5-HT |
| WAY200635 | 5-HT1A |
| L694247 | 5-HT1B, 5-HT1D |
| LY334370 | 5-HT1F |
| DOI | 5-HT2A, 5-HT2B, 5HT2C |
| Ro600175 | 5-HT2B, 5-HT2C |
| RS67506 | 5-HT4 |
| Acetylcholine | M1-M5 |
| Oxotremorine M | M1-M5 |
| McN-A 343 | M1 |
| Adenosine | A1, A2A, A2B, A3 |
| CPA (2-Chloro-N6-cyclopentyl-adenosine) | A1 |
| CGS 21680 | A2A |
| IB-MECA | A3, A1 |
| (−)epinephrine | beta1, beta2, beta3 |
| A 61603 | Alpha1A |
| (R)-(−)-Phenylephrine | Alpha1A, alpha1B, alpha1C |
| Clonidine | alpha2A, 2B, 2C |
| (R)-(+)-m-Nitrobiphenyline | Alpha2C |
| Spermine | Ca-sensing receptor |
| spermidine | Ca-sensing receptor |
| ACEA | CB1 |
| CB65 | CB2 |
| Anadamide | CB2 |
| Dopamine | Dopamine |
| R(+)SKF38393 | D1, D5 |
| Cabergoline | D2, D3, D4, D5 |
| ZK 756326 | CCR8 |
| GABA (g-Aminobutyric acid) | GABAb |
| SKF 97541 | GABAb |
| L-692,585 | ghrelin receptor (GHS-R1a) |
| L-168,049 | glucagon receptor |
| L-glutamate | mGlu |
| L-serine-O-phosphate | mGLU4 |
| L-cysteine sulphonic acid | mGLU3 |
| L-aspartate acid | mGlu |
| Histamine | H1, H2, H3, H4 |
| HTMT dimaleate | H1, H4 |
| Amthamine dihydrobromide | H2 |
| Imetit dihydrobromide | H3, H4 |
| 4-Methylhistamine dihydrochloride | H4 |
| Nicotinic acid | GPR109A |
| Acifran | GPR109A, GPR109B |
| NPPB | GPR35 |
| 17-beta-estradiol | GPR30 |
| ICI182780 | GPR30 |
| Melatonin | MT1, MT2, MT3 |
| ADP | P2Y1, 12, 13 |
| ATP | P2Y2 |
| UTP | P2Y4 |
| UDP | P2Y6 |
| Epoprostenol | IP |
| Prostaglandin D2 | DP |
| Peostaglandin E2 | FP, IP, TP, EP1, 2, 3, 4 |
| Tyramine | TA1, TA2 |
| Pinacidil | KATP |
| SEW 2871 | S1P1 |
| SB 205607 dihydrobromide | delta1 |
| Fentanyl citrate | mu |
| 5-oxo-ETE | OXO receptor |
| Elaidic acid | Free fatty acid |
| LPA | LPA1, LPA2, LPA4 |
| Sphingosine-1-phosphate | S1P1, 2, 3, 4, 5 |
| Oleylethanolamide | GPR119 |
| Leukotriene B4 | BLT1, BLT2 |
| Platelet activating factor PAF (C16) | PAF-R |
| (Trp63, Trp64)-C3a(63-77) | C3A |
| (Tyr65, Phe67)-C5a (65-74) | C5A |
| Angiotensin | AT1, AT2 |
| L162313 | AT1 |
| CGP 42112 | AT2 |
| Apelin | APJ |
| Bombesin | BB1, BB2, BB3 |
| Neuromedin B | BB1 |
| gastrin-releasing peptide | BB2 |
| Bradykinin | B2 |
| Calcitonin | Calcitonin |
| Amylin | Amylin |
| Calcitonin gene-related peptide | CGRP |
| Adrenomedulin | Adrenomedulin |
| CCL7 | CCR1, CCR2, CCR3 |
| CCL3 | CCR1, CCR4, CCR5 |
| CCL20 | CCR6 |
| CCL19 | CCR7 |
| IL8 (CXCL8) | CXCR1 |
| IP10 | CXCR3 |
| A-71623 | CCK1 |
| Cholecystokinin | CCK1, CCK2 |
| Corticotrophin-releasing factor | CRF1, CRF2 |
| Endothelin-1 | ETA, ETB |
| N-Formyl-Met-Leu-Phe | FPR1 |
| WKYMVM | FPR2, FPR3 |
| Galanin | GAL1, GAL2 |
| Ghrelin | Ghrelin |
| Glucagon | Glucagon |
| Glucagon-like peptide | GLP1, GLP2? |
| Secretin | Secretin |
| GRF (ovine) | GHRH |
| GIP (1-39) | GIP |
| Nafarelin | GnRH receptor agonist |
| Kisspeptin 10 | KISS1 receptor |
| Melanin-concentrating hormone | MCH1, MCH2 |
| Alpha-melanocortin (MSH) | MC1, MC2, MC3, M4, MC5 |
| Motilin | MOT |
| Neuromedin U | NMU1, NMU2 |
| Neuropeptide Y | NPY1, Y2, Y4, Y5, Y6 |
| Neuropepide B (NPB-23) | NPBW1, NPBW2 |
| Neurotensin | NTS1, NTS2 |
| Dynorphin A | delta, kappa, mu |
| Nociceptin/orphanin FQ | ORL1 |
| Orexin-A | OX1, OX2 |
| Thyrotropin-releasing hormone | TRH1, TRH2 |
| SFLLR-amide | PAR1 |
| SLIGKV-amide | PAR2 |
| Somatostatin | sst1, 2, 3, 4, 5 |
| Substance P | NK1 |
| Neurokinin A | NK2, NK3 |
| Urotensin II | UT |
| (Arg8)-Vasopressin | V1a, 1b, 2 |
| Vasoactive intestinal peptide (VIP) | VIP |

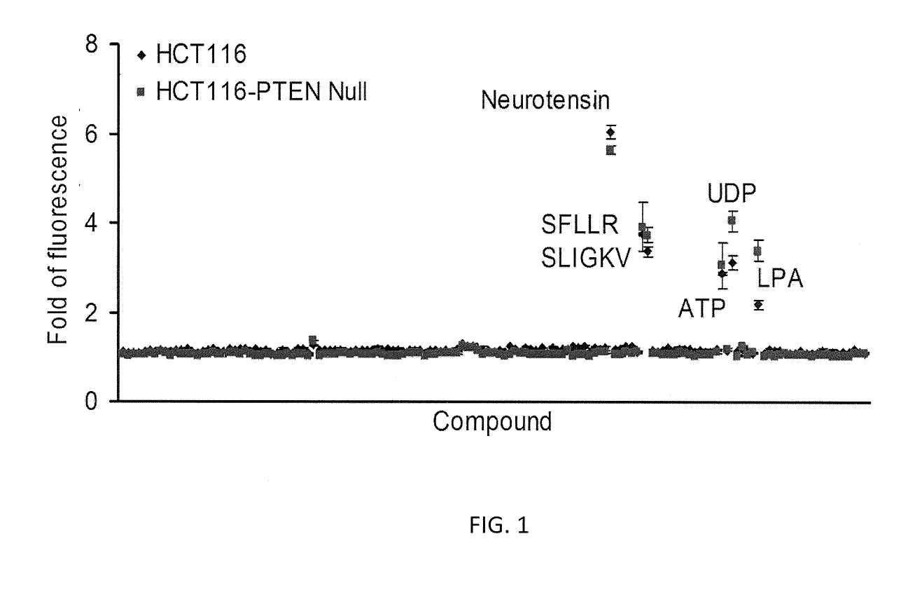

FLIPR to screen endogenous Gq-coupled receptors. FIG. 1 is a graph showing Ca²⁺ mobilization induced by G protein-coupled receptor (GPCR) agonists in the paired isogenic cell lines, PCT116 parental (squares) and HCT116-PTEN Null (diamonds) cell line. The fold increase in fluorescence due to Ca²⁺ mobilization, as measured using FLIPR (Fluorescent Imaging Plate Reader), was plotted as a function of GPCR agonist. Results showed that a small set of agonists caused robust Ca²⁺ mobilization (FIG. 1). These agonists include neurotensin, SFLLR, SLIGKV, ATP, UDP and LPA. The HCT116 cells and the HCT 116-PTEN Null cells responses to treatment with these agonists resulted in pavied data points. That is, there was no clear effect of PTEN deletion on the FLIPR responses induced by these agonists.

### Example 3

### cAMP Screening Using the Paired Isogenic Cell Lines

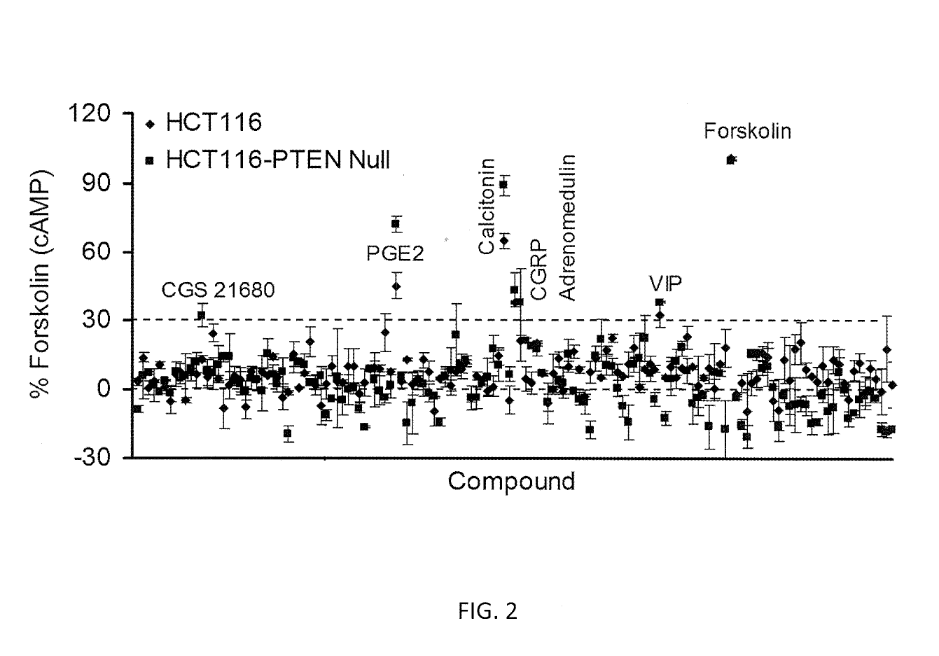

cAMP assays were employed to investigate endogenous Gs-coupled receptors. FIG. 2 is a graph illustrating cAMP accumulation induced by GPCR agonists. The percentage of cAMP level after normalized to the forskolin-induced cAMP increase was plotted as a function of GPCR agonists. PCT116 parental (squares) was compared with HCT116-PTEN Null (diamonds) cell line. Results showed that only a subset of agonists caused detectable cAMP accumulation (FIG. 2) (CGS21680, PGE2, calcitonin, CGRP, adrenomedulin, and VIP). As a control, forskolin also caused significant cAMP accumulation. The PTEN deletion generally increased the cAMP accumulation signals induced by most of these agonists. This cAMP results suggest that PTEN deletion may alter the basal cAMP level.

### Example 4

### DMR Profiling of the Paired Isogenic Cell Lines

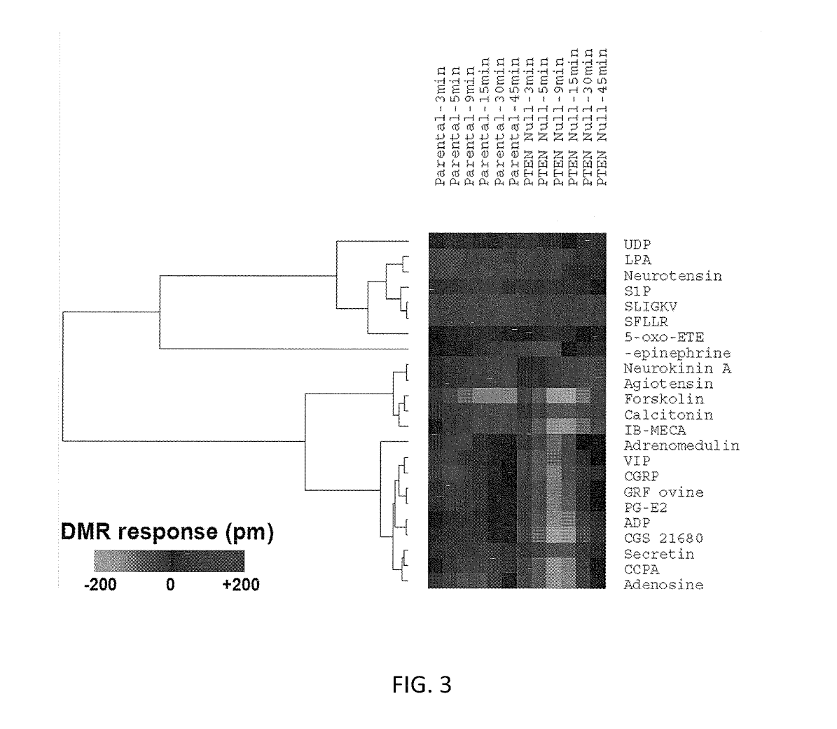

DMR profiles were generated using the same library. FIG. 3 is a heat map of Dynamic Mass Redistribution signals induced by GPCR agonists in PCT116 parental (Parental) and HCT116-PTEN Null (PTEN Null) cell lines. The real responses, as recorded as shift in resonant wavelength (pm), at six time points (3 min, 5 min, 9 min, 15 min, 30 min and 45 min) poststimulation, for each compound-induced DMR was extracted and used as the basis for similarity analysis. The contrast was set to be −200 to 200 pm. The saturated (black) areas mean a response whose absolute value is equal to and greater than 200 pm. The heat map was generated using unsupervised Ward hierarchical clustering. Only DMR signal that contains at least one data point greater than 80 pm was included in the analysis. Results showed that although 22 different agonists, as well as the control compound forskolin, triggered robust DMR signals with an amplitude greater than 80 picometer (FIG. 3). These results suggest that (1) DMR assays have sensitivity to probe endogenous GPCRs; and (2) DMR assays can easily differentiate the cellular responses induced by these agonists.

### Example 5

### Quantitative RT-PCR of the Paired Isogenic Cell Lines

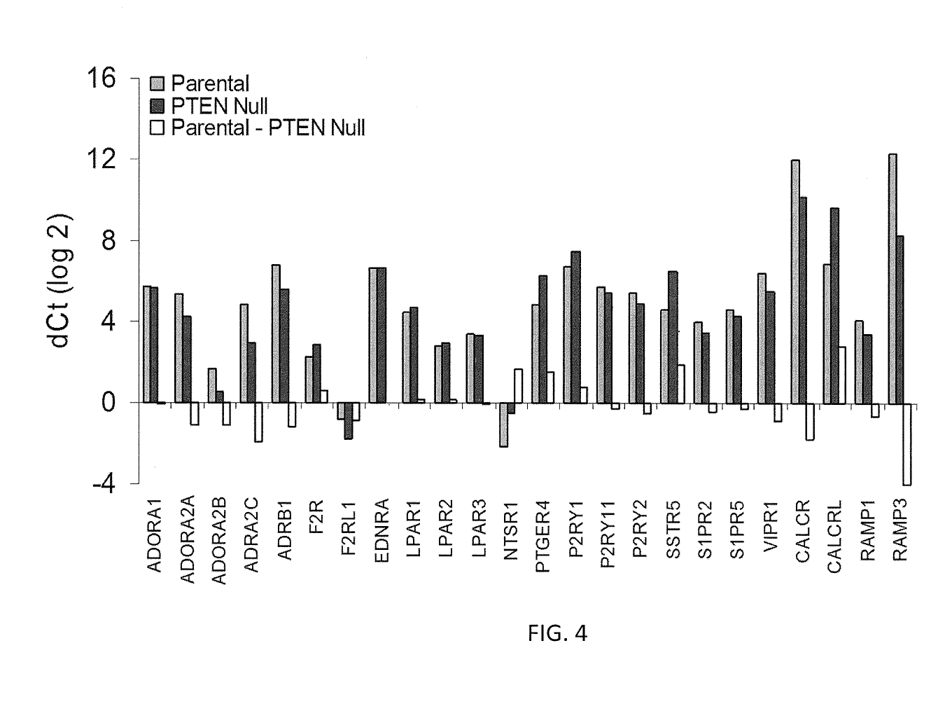

Next, we performed expression analysis of 352 GPCRs in the paired isogenic cell lines using whole plate based quantitative real time PCR. These results are shown in FIG. 4. FIG. 4 is a graph showing quantitative real time PCR confirmed that the expression pattern of a panel of GPCRs is largely identical in the two isogenic cell lines. Almost all GPCRs, had mRNA levels which were found to be within 2 fold after PTEN deletion. For each cell line, the cycle threshold value of each gene was normalized to the control gene, Hypoxanthine phosphoribosyltransferase 1. Results showed that only a subset of GPCR genes were expressed at relatively high level. Among these receptors, a large number of receptors have known agonists reported in literature. Comparative analysis suggest that the gene expression pattern between the paired isogenic cell lines is largely the same, and PTEN deletion has little effect on the gene expression of these receptors (FIG. 4). This result also suggests that the great difference in the DMR signals of the panel of agonists detected, as shown in FIG. 3, is due to the alteration of signaling pathway downstream from these receptors, rather than the gene expression level of the receptors being activated.

### Example 6

### Pathway Deconvolution of the Agonist-Induced DMR Signals in the Isogenic Cell Lines

Next, we used chorea toxin and pertussis toxin to deconvolute the signaling pathways being activated by these agonists. FIG. 5 a, b and c are graphs illustrating DMR characteristics of 10 μM adenosine in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). FIG. 5 summarized the results for the adenosine-induced DMR. RT-PCR (as shown in FIG. 4) study showed that HCT116 cell lines predominately express adenosine A2B receptor. Adenosine triggered a DMR signal that is greatly different in the PTEN deleted HCT116 cell line, compared to that in the parental cell line (FIG. 5a). The permanent activation of Gs protein by CTx almost completely suppressed the adenosine DMR, but uncoupled Gi protein by PTx only accelerated the adenosine DMR in both cell lines (FIG. 5b and c, respectively). These results suggest that adenosine primarily activates endogenous Gs-coupled adenosine A2B receptors in both the HCT116 cell line and the PTEN deleted HCT 116 cell line.

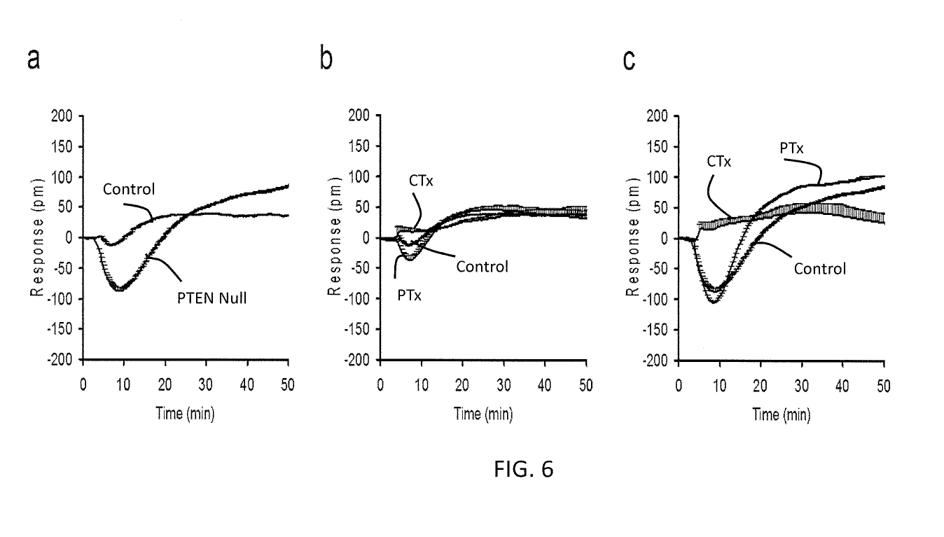

FIG. 6 summarized the results for the epinephrine-induced DMR. FIG. 6 a, b and c are graphs illustrating DMR characteristics of 10 μM epinephrine in the two isogenic cell lines (HCT116 parental cell line; HCT116-PTEN Null cell line) under different conditions: (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). RT-PCR (as shown in FIG. 4) study showed that HCT116 cell lines predominately express beta1-adrenergic receptor. Epinephrine triggered a DMR signal that greatly differed in the PTEN deleted HCT116 cell line, compared to that in the parental cell line (FIG. 6a). The permanent activation of Gs protein by CTx almost completely suppressed the adenosine DMR, but uncoupled Gi protein by PTx had little effect on the epinephrine DMR in both cell lines (FIGS. 6b and c, respectively). These results suggest that epinephrine primarily activates endogenous Gs-coupled beta1-adrenergic receptor.

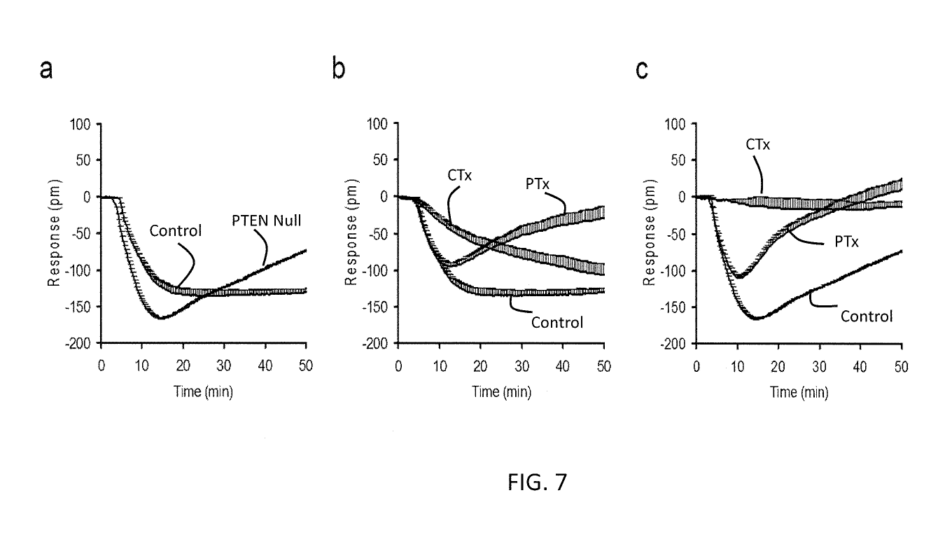

FIG. 7 summarized the results for the forskolin-induced DMR. FIG. 7 a, b and c are graphs illustrating DMR characteristics of 10 μM forskolin in the two isogenic cell lines (HCT116 parental cell line; HCT116-PTEN Null cell line) under different conditions: (a) untreated cell lines; (b) HCT 116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). Forskolin is an activator of adenylyl cyclases, whose activation leads to cAMP-PKA pathway. As expected, forskolin triggered a DMR similar to both adenosine and epinephrine. Furthermore, similar to epinephrine and adenosine, PTEN deletion also affected the forskolin induced DMR, further suggesting that PTEN deletion primarily affected the signaling pathways, rather than the expression of receptors. The permanent activation of Gs protein by CTx almost completely suppressed the forskolin DMR in both cell lines, but uncoupled Gi protein by PTx only accelerated the forskolin DMR (FIGS. 7b and c, respectively). These results suggest that forskolin triggers signaling primarily via cAMP-PKA pathway.

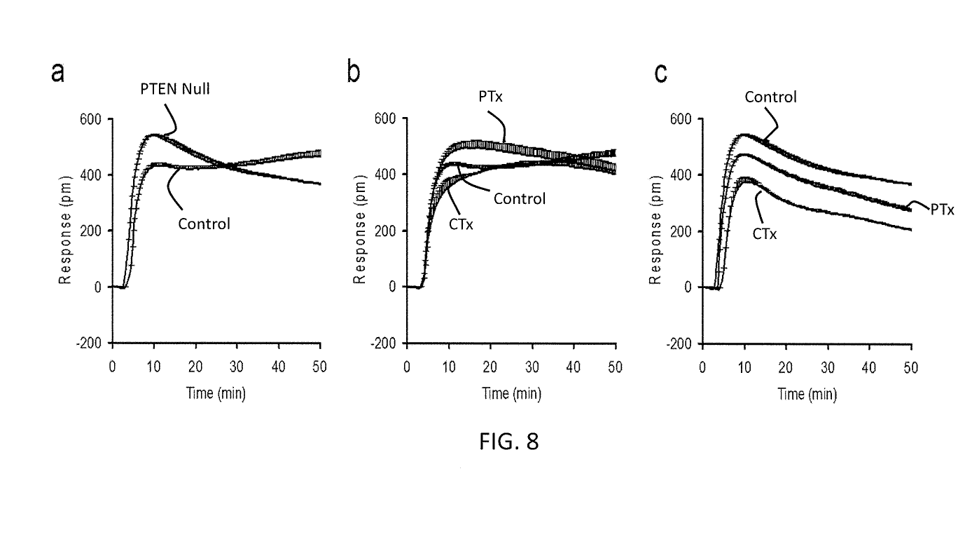

FIG. 8 summarized the results for the SFLLR-induced DMR. FIG. 8 a, b and c are graphs illustrating DMR characteristics of 10 μM SFLLR in the two isogenic cell lines (HCT116 parental cell line; HCT116-PTEN Null cell line) under different conditions: (a) untreated cell lines; (b) HCT 116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). SFLLR is a synthetic peptide agonist for PAR1. RT-PCR (as shown in FIG. 4) study showed that HCT116 cell lines express both PAR1 and PAR2 at relatively high levels. SFLLR triggered a robust DMR signal in both cell lines, but with distinct characteristics (FIG. 8a). However, CTx pretreatment led to a modulation profile different from the PTx pretreatment in the paired cell lines, suggesting that the alteration of PI3K pathway affects the PAR1-mediated signaling.

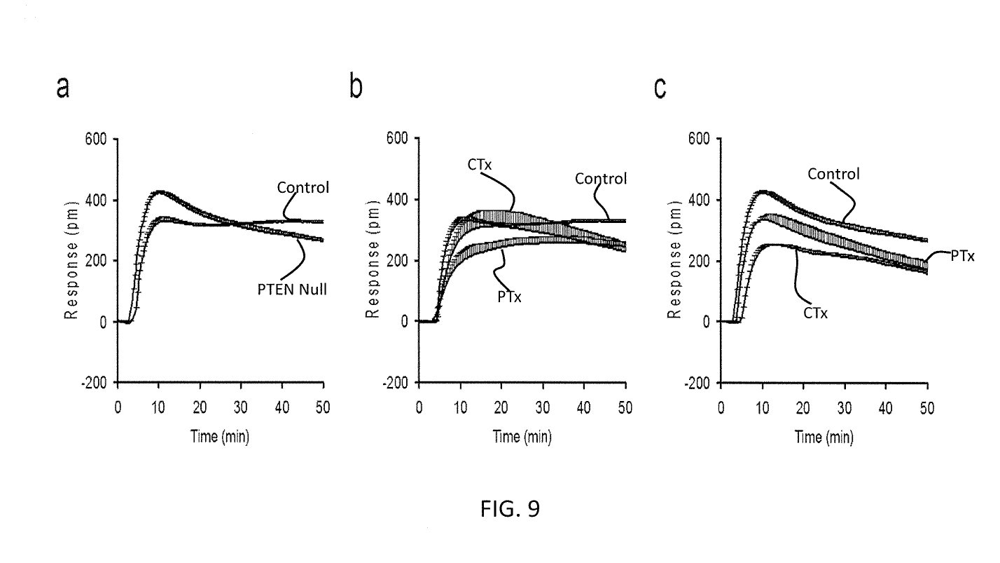

FIG. 9 summarized the results for the SLIGKV-induced DMR. FIG. 9 a, b and c are graphs illustrating DMR characteristics of 10 μM SLIGKV in the two isogenic cell lines (HCT116 parental cell line; HCT116-PTEN Null cell line) under different conditions: (a) untreated cell lines; (b) HCT 116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). SLIGKV is a synthetic peptide agonist for PAR2. RT-PCR (as shown in FIG. 4) study showed that HCT116 cell lines express both PAR1 and PAR2 at relatively high levels. The modulation patterns obtained using CTx and PTx closely resembled their effects on the SFLLR DMR.

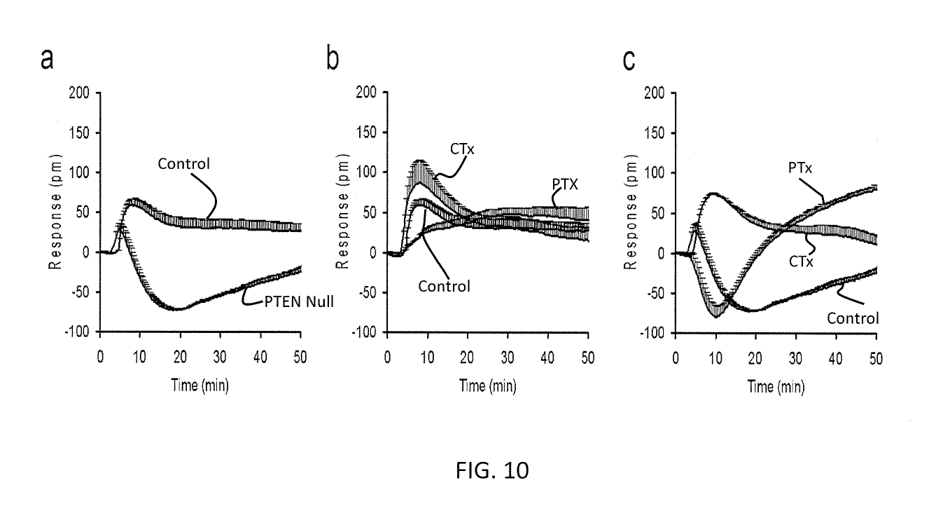

FIG. 10 summarized the results for the ATP-induced DMR. FIG. 10 a, b and c are graphs illustrating DMR characteristics of 10 μM ATP in the two isogenic cell lines (HCT116 parental cell line and PTEN Null cell line) under different conditions. FIG. 10 (a) untreated cell lines; (b) HCT 116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4). RT-PCR (as shown in FIG. 4) study showed that HCT116 cell lines express multiple P2Y receptors, including P2Y1, P2Y2 and P2Y11, all of which are Gq-coupled receptors, except for that P2Y11 is also coupled to Gs pathway. The dramatically different DMR in the PTEN-deleted cell line suggests that PTEN deletion altered the signaling pathways of P2Y receptors activated by ATP. This is confirmed by toxin treatment. Together, these results suggest that ATP activates multiple GPCRs and mediates signaling via multiple G proteins in both cell lines.

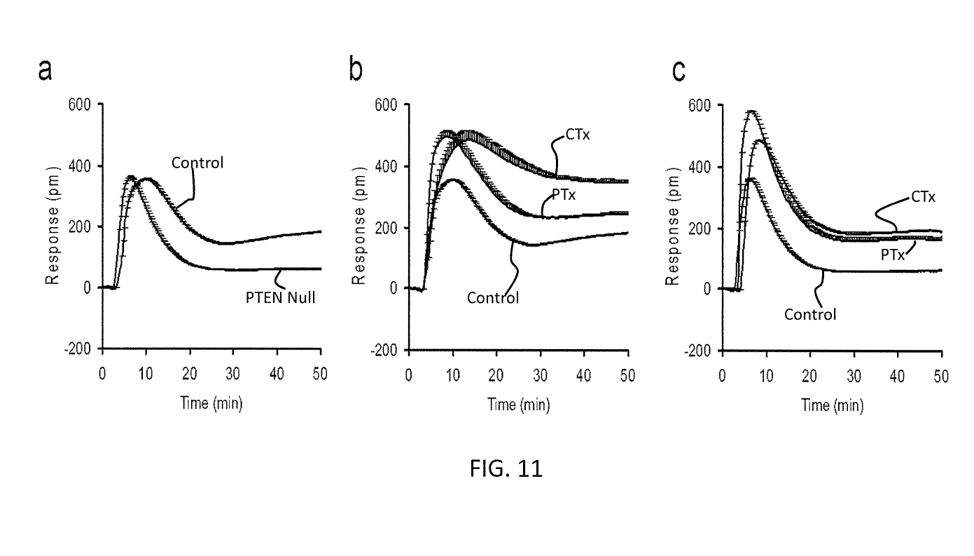

FIG. 11 summarizes the results for neurotensin-induced DMR. FIG. 11 a, b and c are graphs illustrating DMR characteristics of 10 μM neurotensin in the two isogenic cell lines (HCT116 parental cell line, HCT116-PTEN Null cell line) under different conditions. FIG. 11(a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Results show that neurotensin activated endogenous NTS1 receptor but mediated signaling via multiple pathways in both cell lines.

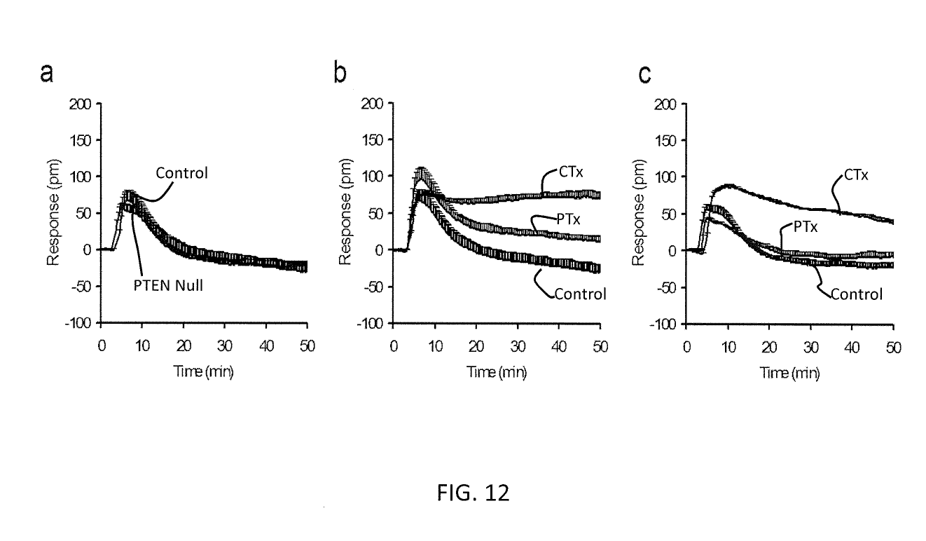

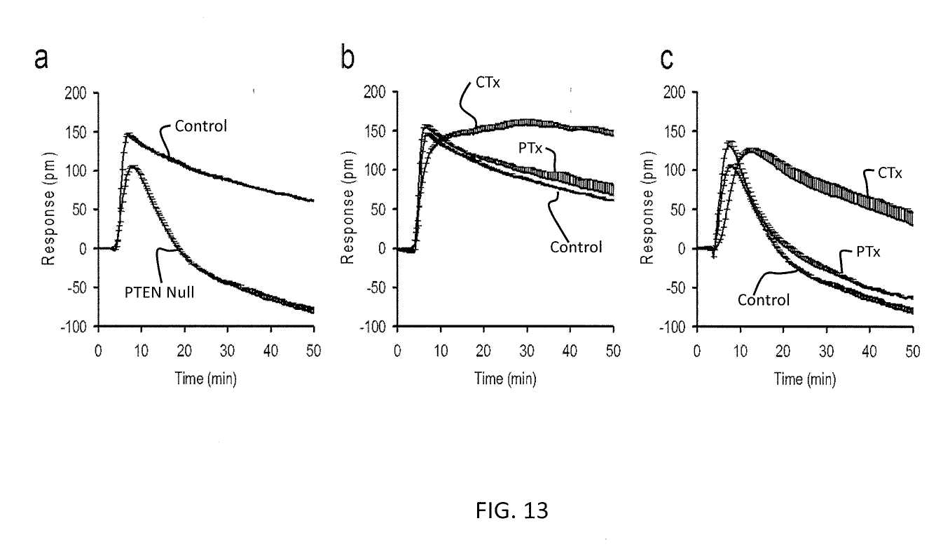

By comparison, we also investigated the DMR signals of two growth factors, EGF and IGF1 (FIG. 12 and FIG. 13, respectively). Toxin treatment also led to similar patterns for these two growth factor-mediated signals. Interestingly, EGF DMR was insensitive to PTEN deletion. However, PTEN deletion has clear effects on the IGF1-induced DMR.

FIG. 12 summarizes the results for EGF-induced DMR. FIG. 12 a, b and c are graphs illustrating DMR characteristics of 1 μM epidermal growth factor (EGF) in the two isogenic cell lines (HCT116 parental cell line; HCT116-PTEN Null cell line) under different conditions. FIG. 12 (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

FIG. 13 summarizes the results for IGFI. FIG. 13 a, b and c are graphs illustrating DMR characteristics of 1 μM insulin growth factor-1 (IGF1) in the two isogenic cell lines (HCT116 parental cell line; HCT116-PTEN Null cell line) under different conditions. FIG. 13 (a) untreated cell lines; (b) HCT116 parental cells without (Control) or with cholera toxin (CTx) or pertussis toxin (PTx) pretreatment; (c) HCT116-PTEN Null cells without (Control) or with cholera toxin (CTx) or pertussis toxin (PTx) pretreatment. Data represents mean±s.d. from duplicate measurements (n=4).

### Example 7

### Western Blot Analysis of Signaling Pathways Activated by Five Different GPCR Agonists

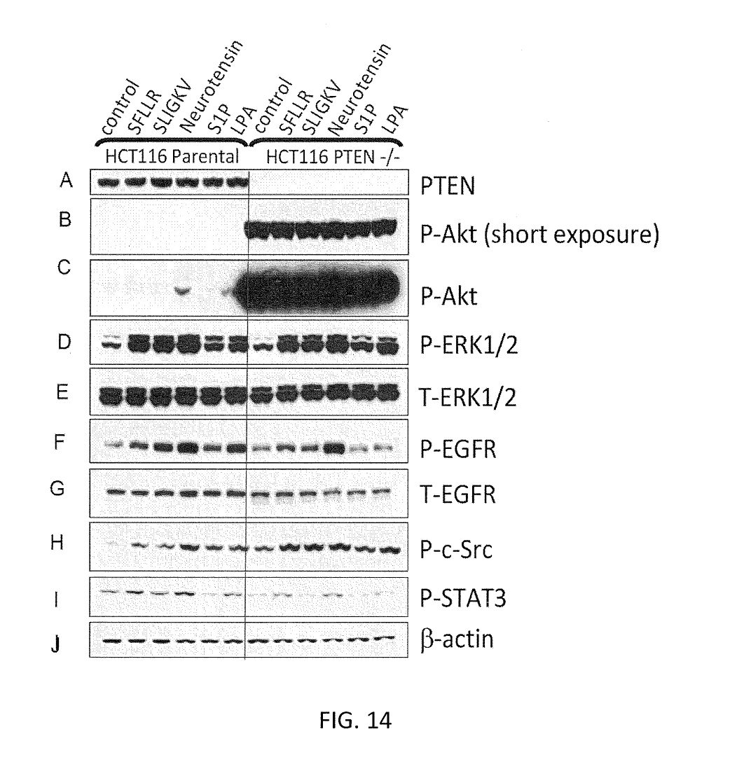

To study the signaling pathways activated by different GPCR agonists, we performed Western blot analysis of key signaling proteins in GPCR signaling. FIG. 14 is a photograph of a Western blot. FIG. 14 illustrates that multiple pathways are transactivated by multiple GPCR agonists. Cells were serum-starved overnight and then treated with each of 5 GPCR agonists for 5 minutes in serum-free medium. Proteins in the cell lysates were resolved on 4-12% SDS-PAGE gel, followed by Western blotting with each of these indicated antibodies. SFM=serum starvation overnight (0% serum) and serum-free medium for the experiment. Compound treatment: 5 min. The concentration for each compound was SFLLR=1 μM, SLIGKV=1 μM, Neurotensin=μuM, S1P=2.5 μM, and LPA=2.5 μM. P=phospho and T=total in the labels. Results showed that PTEN null cells did not express PTEN, whereas the parental cells expressed PTEN (FIG. 14, Panel A). In the loss of PTEN that dephosphorylates the PIP3, PI3K produces more PIP3 which in turn activates Akt kinase. FIG. 14 Panel B & C demonstrate that the basal activity of Akt in the control condition was much higher in the PTEN null cells than in HCT116 parental cells where Akt activation was barely detectable in the control condition. Except that two agonists (neurotensin and LPA), all other three agonists (SFLLR, SLIGKV, and S1P) did not activate the Akt both in the parental and PTEN null cells. GPCRs can transactivate ERK1/2 kinases and receptor tyrosine kinases. FIG. 14 Panel D & E show that the basal ERK1/2 activity and total ERK1/2 are similar in the HCT116 parental and PTEN null cells. All five agonists robustly activated ERK1/2 in this pair of cell lines, whereas there was no significant difference seen between the parental and PTEN null cells. The basal EGFR activity and EGFR level are similar between the parental and PTEN null cell lines. All five agonists activated variably EGFR in the parental cells, whereas S1P and LPA did not activate EGFR in the PTEN null cells (FIG. 14 Panel F and G). The basal activity of c-Src was much higher in the PTEN null cells, compared with the parental cells (FIG. 14 Panel H). The basal activity of STAT3 was lower in the PTEN null cells, compared to the parental cells (FIG. 14 Panel I). Further, the stimulation of both cell lines with SFLLR and neurotensin significantly increased the level of phosphorylated STAT3 (FIG. 14 Panel I). As a control, the beta-actin level was almost identical for all samples obtained from either the parental cells, or the PTEN null cells (FIG. 14 Panel J). All five agonists further activated c-Src in both of parental and PTEN null cells.

### Example 8

### Dose Responses of SFLLR in the Paired Isogenic Cell Lines

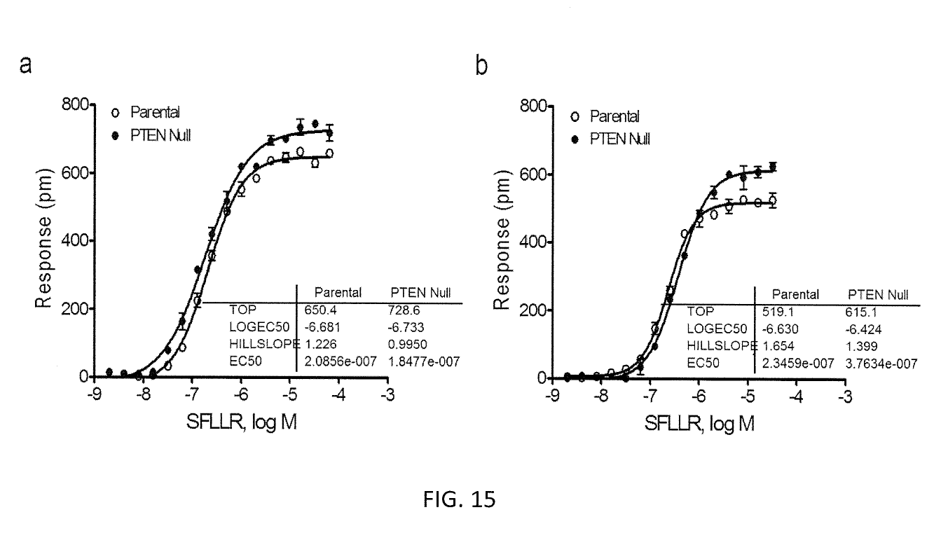

Next, we characterized the pharmacology of SFLLR in the paired isogenic cell lines using DMR assays. FIG. 15 a and b are graphs illustrating differences in potency and efficacy of SFLLR to trigger DMR signals in between the two isogenic cell lines. Both the early DMR amplitude (a) and the late DMR amplitude (b) were plotted as a function of SFLLR doses. The dose response suggests that the potency of SFLLR to trigger DMR, based on either the early positive-DMR (P-DMR) or the late P-DMR (50 min poststimulation) amplitudes, was almost identical (FIG. 15). However, the efficacy of SFLLR was distinct in the paired isogenic cell lines—SFLLR gave rise to higher efficacy in the PTEN null cell line, based either the early or the late PDMR event. This suggests that PTEN deletion did not alter the protein level of PAR1; rather, it altered the signaling pathways downstream PAR1, leading to enhanced signaling capacity upon the activation of PAR1 by SFLLR.

### Example 9

### DMR Characteristics of a Panel of Kinase Inhibitor Drugs in the Paired Isogenic Cell Lines

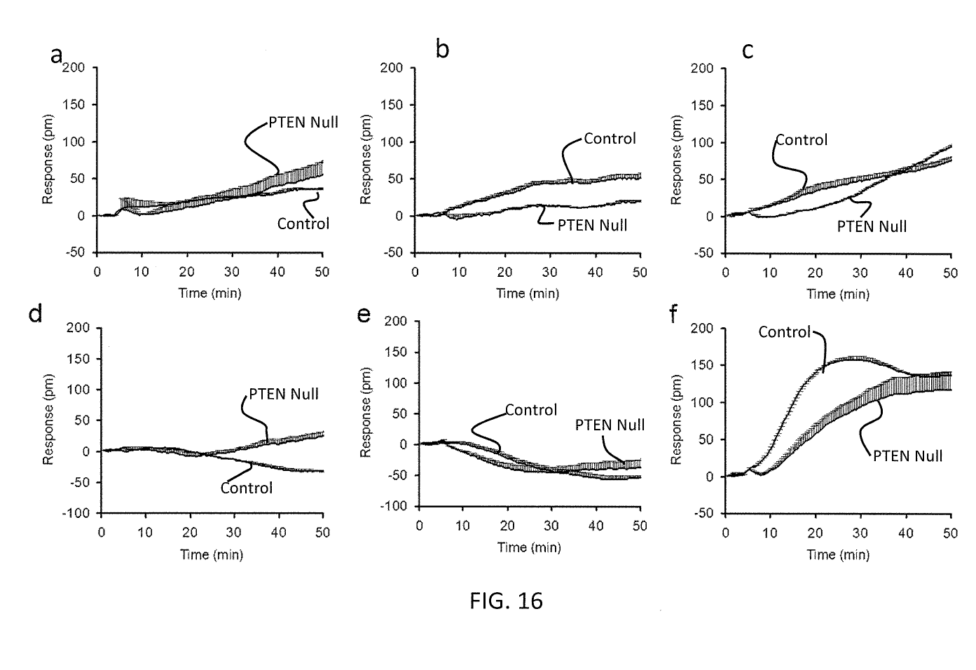

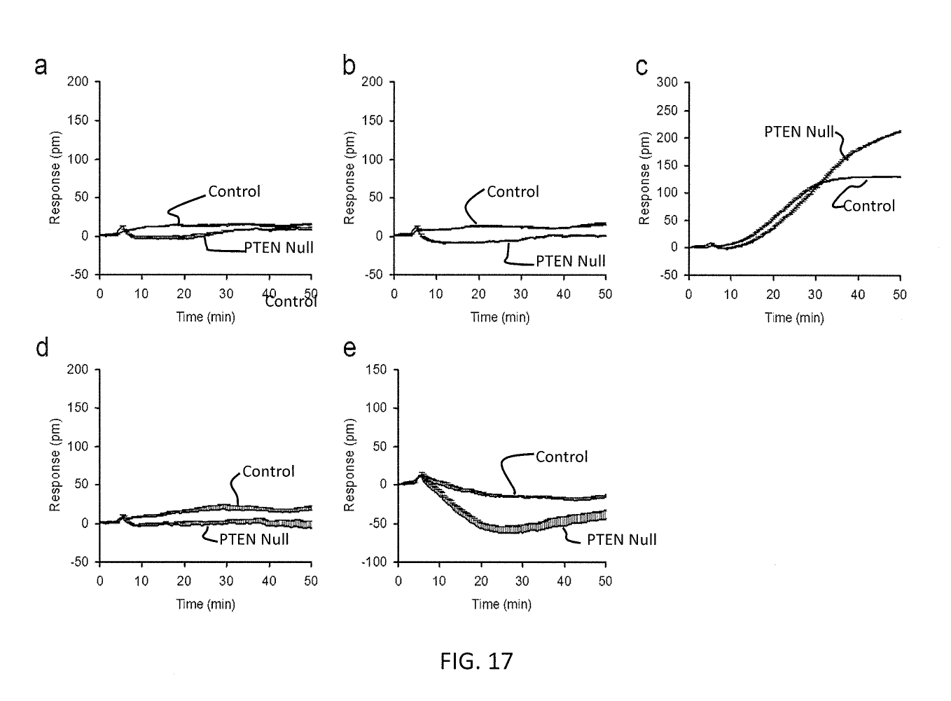

Next, we characterized 11 FDA approved kinase inhibitor drugs in the paired isogenic cell lines using DMR assays. Results are summarized in FIGS. 16 and 17. FIG. 16 a, b, c, d, e and f are graphs illustrating DMR characteristics of a panel of kinase inhibitor drugs in both HCT116 parental (Control) cell line and HCT116-PTEN null (PTEN null) cell lines. (a) Everolimus, (b) Sirolimus, (c) Temsirolimus, (d) Erlotinib, (e) Geftinib, and (f) Imatinib. Each was assayed at 16 μM. The data represents mean±s.d. from 2 independent measurements, each in duplicate (n=4). FIG. 17 a, b, c, d and e are graphs illustrating DMR characteristics of a panel of kinase inhibitor drugs in both HCT116 parental (Control) cell line and HCT116-PTEN null (PTEN null) cell lines. (a) lapitinib, (b) pazopanib, (c) nilotinib; (d) sorafenib, (e) sunitinib. Each was assayed at 16 μM. The data represents mean±s.d. from 2 independent measurements, each in duplicate (n=4). Results showed that different kinase inhibitor drugs triggered different DMR signals in the parental cell line, and their DMR characteristics were often sensitive to the PTEN deletion. Interestingly, the three mTOR inhibitor drugs, everolimus, sirolimus, and temsirolimus, behaved similarly. All triggered detectable DMR with similar characteristics in the parental cell line. PTEN deletion generally suppressed or decreased the kinetics of their P-DMR signal for m-TOR inhibitors. The EGFR inhibitor drugs, gefitinib and erlotinib gave rise to very small DMR signals in either cell line. The two BCR-ABL inhibitor drugs imatinib and nilotinib, led to similar DMR that was sensitive to PTEN deletion.

The HER2 inhibitor drug lapatinib did not result in any detectable DMR in both cell lines; neither did VEGFRs and PDGFR dual inhibitor drugs pazopanib and sorafenib. However, another multi-kinase inhibitor drug sunitinib led to a DMR that was sensitive to PTEN deletion. Together, these results suggest that the phenotypic profiles using DMR assays can detect the differential sensitivity of these kinase drugs in the paired isogenic cell lines.

### Example 10

### DMR Characteristics of a Panel of Kinase Inhibitor Drugs in the Paired Isogenic Cell Lines Using Reporters

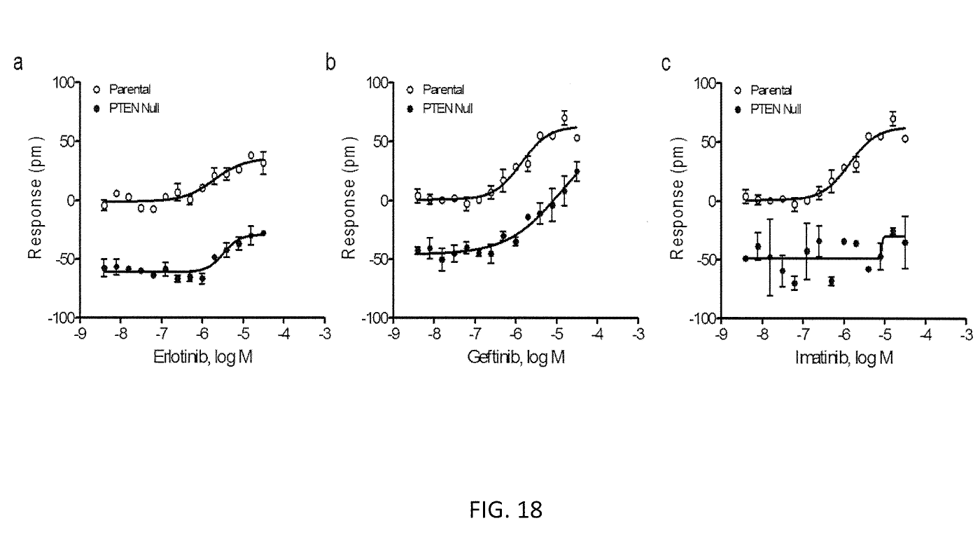

Next, we characterized 11 FDA approved kinase inhibitor drugs in the paired isogenic cell lines using DMR reporters. FIG. 18 a, b and c are graphs illustrating the dose-dependent alteration of the late DMR of IGF1 in the two isogenic cell lines by different kinase inhibitors: (a) erlotinib; (b) geftinib; (c) imatinib. The DMR amplitudes 50 min postsimulation with 2 nM IGF1 were plotted as a function of kinase inhibitors. The kinase inhibitors at different doses were used to pretreat the cells for about 1 hr, before IGFR1 was added. The DMR reporter used was IGF1. IGF1 triggered a robust DMR signal in the paired isogenic cell lines, with greater sensitivity to the PTEN deletion (FIG. 13). The pretreatment of cells with different kinase inhibitor drugs at different doses gave rise to distinct patterns of modulation of the IGF1-induced DMR (FIG. 18). Both erlotinib and geftinib had little impact on the early P-DMR amplitude of the IGF1 DMR; however, both increased in the dose-dependent manner the late response (i.e., signal at 50 min poststimulation) of the IGF1 DMR in both cell lines. Furthermore, their potency to increase the late IGF1 response was lower in the PTEN-deleted cell line (FIGS. 18a and b). These results suggest that when the kinase inhibitors can be studied using DMR profiling of kinase inhibitor drugs alone, one can use a specific reporter to manifest their mode of action.

In contrast, imatinib only dose-dependently increased the late IGF1 DMR response in the parental cells, but not the PTEN deleted cells. This is consistent with the great sensitivity of imatinib-induced DMR to the PTEN deletion.

### Example 11

### DMR Phenotypic Classification of Cancer Cell Lines

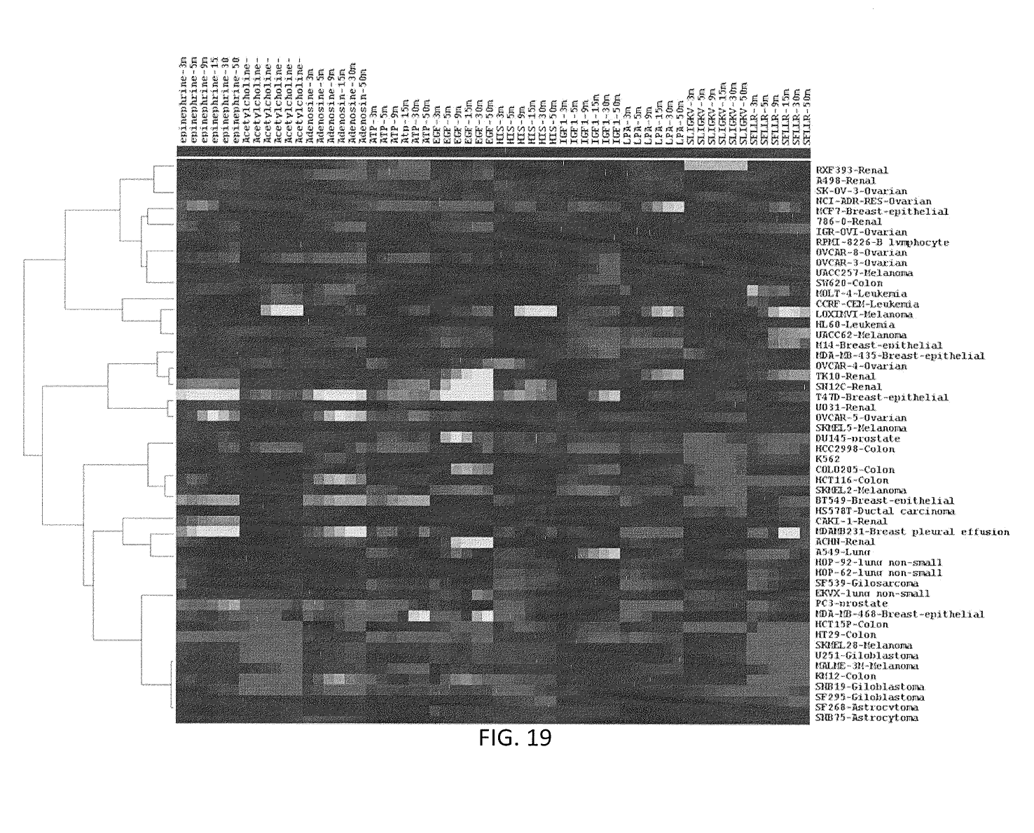

Next, we performed classification analysis of a large panel of cancer cell lines using DMR profiles of a predetermined panel of receptor agonists. The receptor agonist panel includes epinephrine, acetylcholine, adenosine, ATP, EGF, histamine (HIS), IGF1, LPA, SLIGKV, and SFLLR. The cell lines are 54 cell lines from NCI60 panel. Cluster analysis, based on the DMR signals of these agonists in respective cell lines, led to a high resolution classification of these cell lines into distinct clusters (FIG. 19).

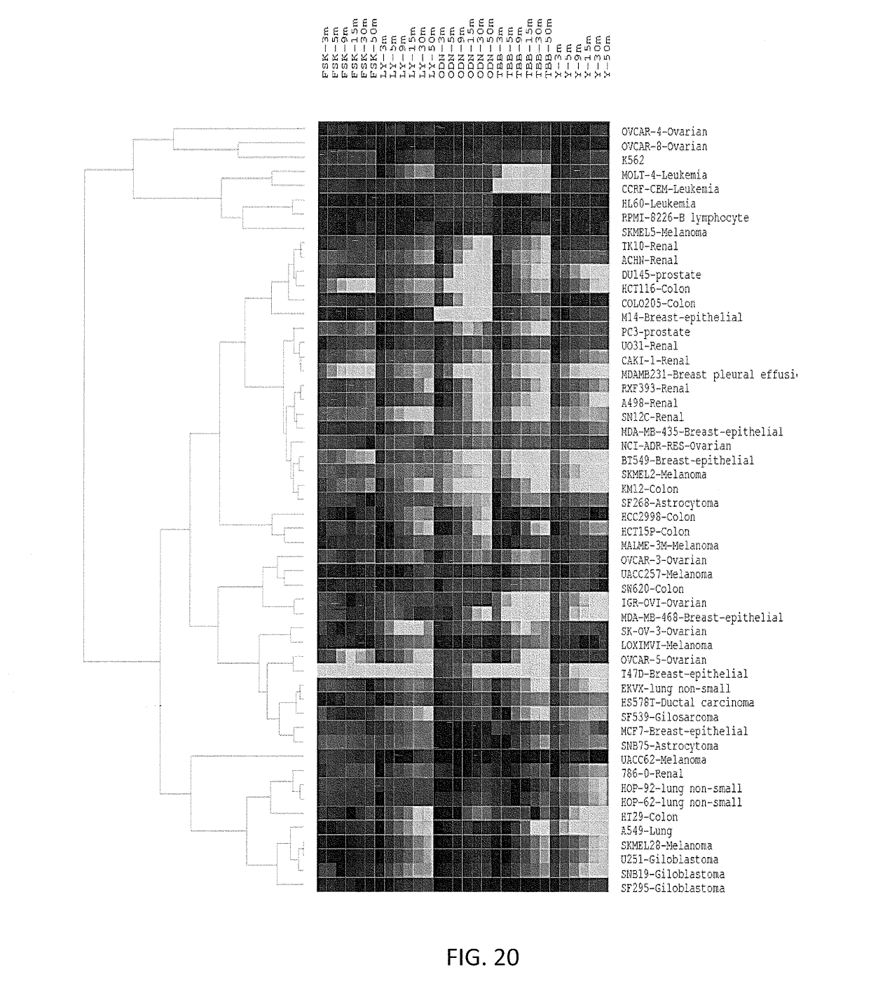

We also performed classification analysis of the same panel of cancer cell lines using DMR profiles of a predetermined panel of reference compounds for distinct intracellular targets. The reference compounds include the adenylyl cyclase activator forskolin (FSK), the PI3K inhibitor LY294002 (LY), the TLR9 agonist ODN2006 (ODN), the casein kinase 2 inhibitor TBB, and the ROCK inhibitor Y27632 (Y) (FIG. 20). The cell lines are the same 54 cell lines from NCI60 panel. Cluster analysis, based on the DMR signals of these agonists in respective cell lines, led to a high resolution classification of these cell lines into distinct clusters that were different from those obtained using cell surface receptor agonist panel.

In summary, these results suggest that it is possible to differentiate cancer cells of different origins, and differentiate the drug responses using the methods disclosed in this document.

We claim:

1. A method of using a label-free biosensor for cancer diagnostics and chemotherapy selection comprising:
a. providing an array of resonant waveguide grating biosensors;
b. culturing one or more cancer cells on at least one biosensor in the biosensor array;
c. treating the cultured cancer cell with at least one chemotherapeutic agent in the absence and presence of at least one agonist;
d. Collecting dynamic mass redistribution (DMR) signals from the cultured cell treated with at least one chemotherapeutic agent in the absence and presence of the at least one agonist in the biosensor array; and,
e. performing comparative analysis to generate a cancer cell fingerprint of the cancer cell responding to the at least one chemotherapeutic agent in the absence and presence of the at least one agonist.

2. The method of claim 1 wherein the at least one agonist is selected from the group consisting of an Gq-coupled receptor agonist, a Gs-coupled receptor agonist, a Gi-coupled receptor agonist, a growth factor receptor agonist.

3. The method of claim 2 wherein the Gq-coupled receptor agonist is histamine.

4. The method of claim 2 wherein the Gq-coupled receptor agonist is acetylcholine.

5. The method of claim 2 wherein the Gs-coupled receptor agonist is epinephrine.

6. The method of claim 2 wherein the Gi-coupled receptor agonist is dopamine.

7. The method of claim 1 wherein the cancer cell is an immortalized cancer cell.

8. The method of claim 2 wherein the cancer cell is an immortalized cancer cell.

9. The method of claim 1 wherein the cancer cell is a primary cell.

10. The method of claim 2 wherein the cancer cell is a primary cell.

11. The method of claim 1 wherein the cancer cell is a primary cell derived from a solid tumor.

12. The method of claim 2 wherein the cancer cell is a primary cell derived from a solid tumor.

13. The method of claim 1 further comprising culturing one or more reference cells on at least one biosensor in the biosensor array, wherein the reference cell generates a reference cell fingerprint.

14. The method of claim 13 further comprising comparing the reference cell fingerprint to the cancer cell fingerprint.

15. The method of claim 1 further comprising treating the cultured cell with at least one reference compound, wherein the reference compound generates a reference compound control fingerprint.

16. The method of claim 14 wherein the reference compound is selected from the group consisting of forskolin, 4,5,6,7-tetrabromobenzotriazole, LY294002 and ODN2006.

17. The method of claim 14 further comprising comparing the cancer cell fingerprint with the reference compound control fingerprint.

18. The method of claim 1 further comprising using the cancer cell fingerprint to determine a chemotherapy regimen to treat the cancer represented by the cancer cells.

19. A method of using a label-free biosensor for cancer diagnostics and chemotherapy selection comprising:
a. providing an array of resonant waveguide grating biosensors;
b. culturing a cancer cell on at least one biosensor in the biosensor array;
c. treating the cultured cancer cell with at least one chemotherapeutic agent in the absence and presence of at least one agonist;
d. Collecting dynamic mass redistribution (DMR) signals from the cultured cancer cell treated with at least one chemotherapeutic agent in the absence and presence of the at least one agonist in the biosensor array;
e. performing comparative analysis to generate a cancer cell fingerprint of the cultured cancer cell responding to the at least one chemotherapeutic agent in the absence and presence of the at least one agonist;
f. culturing a reference cell on at least one biosensor in the biosensor array, treating the cultured reference cell with the at least one chemotherapeutic agent in the absence and presence of the at least one agonist, collecting the DMR signals from the cultured reference cell treated with the at least one chemotherapeutic agent in the absence and presence of the at least one agonist in the biosensor array, so the cultured reference cell generates a reference cell fingerprint responding to the at least one chemotherapeutic agent in the absence and presence of the at least one agonist;
g. comparing the reference cell control fingerprint to the cancer cell fingerprint;
h. treating the cancer cell and the reference cell with at least one reference compound, wherein the reference compound generates a reference compound control fingerprint;
i. comparing the cancer cell fingerprint with the reference cell control fingerprint and the reference compound control fingerprint; and,
j. using the compared data from the cancer cell fingerprint, the reference cell fingerprint and the reference compound control fingerprint to determine a chemotherapy regimen to treat the cancer represented by the cancer cells.
